SEQUENCE LISTING (21 sequences)

SEQ ID NO: 1 - protein, 5 aa, synthetic construct
GLMVA

SEQ ID NO: 2 - protein, 5 aa, synthetic construct
GLAVM

SEQ ID NO: 3 - protein, 21 aa, synthetic construct
EAIEGTYIDKKCPFTGNVSIR

SEQ ID NO: 4 - protein, 22 aa, synthetic construct
ELDLSNNCLGDAGILQLVESVR

SEQ ID NO: 5 - protein, 27 aa, synthetic construct
ITAFVPNDGCLNFIEENDEVLVAGFGR

SEQ ID NO: 6 - protein, 27 aa, synthetic construct
GLYGIKDDVFLSVPCILGQNGISDLVK

SEQ ID NO: 7 - protein, 22 aa, synthetic construct
ELDLSNNCLGDAGILQLVESVR

SEQ ID NO: 8 - protein, 19 aa, synthetic construct
YAICSALAASALPALVMSK

SEQ ID NO: 9 - protein, 14 aa, synthetic construct
RSIQFVDWCPTGFK

SEQ ID NO: 10 - protein, 4 aa, synthetic construct
GFAF

SEQ ID NO: 11 - protein, 4 aa, synthetic construct
GFAF

SEQ ID NO: 12 - protein, 4 aa, synthetic construct
GFAF

SEQ ID NO: 13 - protein, 4 aa, synthetic construct
PFAF

SEQ ID NO: 14 - protein, 4 aa, synthetic construct
PFAF

SEQ ID NO: 15 - protein, 4 aa, synthetic construct
FPAF

SEQ ID NO: 16 - protein, 4 aa, synthetic construct
APFF

SEQ ID NO: 17 - protein, 4 aa, synthetic construct
FAFA

SEQ ID NO: 18 - protein, 4 aa, synthetic construct
FAFA

SEQ ID NO: 19 - protein, 4 aa, synthetic construct
FPAF

SEQ ID NO: 20 - protein, 4 aa, synthetic construct
APFF

SEQ ID NO: 21 - protein, 5 aa, synthetic construct
SLGTC

(19) United States
(12) Patent Application Publication
(10) Pub. No.: US 2026/0235617 A1
(43) Pub. Date: Aug. 13, 2026

(54) MULTIPLEXED SINGLE CELL PROTEOMICS

(71) Applicants: University of Washington, Seattle, WA (US); THE REGENTS OF THE UNIVERSITY OF CALIFORNIA, Oakland, CA (US)

(72) Inventors: Brian BELIVEAU, Seattle, WA (US); Devin SCHWEPPE, Seattle, WA (US); Yuzhen LIU, Seattle, WA (US); Christopher MCGANN, Seattle, WA (US); Erik J. BERGSTROM, Seattle, WA (US); Keriann Marie BACKUS, Seattle, WA (US)

(21) Appl. No.: 19/153,695

(22) PCT Filed: Feb. 5, 2024
(86) PCT No.: PCT/US2024/014448

§ 371 Date: Aug. 5, 2025

Related U.S. Application Data
(60) Provisional application No. 63/483,598, filed on Feb. 7, 2023.

(51) Int. Cl. G01N 33/68; C07K 7/06; G01N 33/58

(52) U.S. Cl. CPC G01N 33/6842; C07K 7/06; G01N 33/58; G01N 2570/00

(57) ABSTRACT

Disclosed are functionalized labels, labeled proteins, and methods for labeling, measuring, detecting and visualizing proteins. The methods can be used for quantitative single-cell proteomics, combinatorial indexing, and multiplexing proteomics.

### CROSS REFERENCES

This application claim priority to U.S. Provisional Patent Application Ser. No. 63/483,598, filed Feb. 7, 2023, incorporated by reference here in its entirety.

### SEQUENCE LISTING STATEMENT

A computer readable form of the Sequence Listing is filed with this application by electronic submission and is hereby incorporated by reference in its entirety. The Sequence Listing is contained in the XML file created on Feb. 1, 2024 having the name “22-0096-WO.xml” and is 19,156 bytes in size.

### BACKGROUND

No two cells are identical. Therefore, understanding the unique features and functions of each cell of an organism is key to understanding what defines multicellular life. Breakthroughs in nucleic acid sequencing technology have revolutionized biology by providing global portraits of how DNA and RNA vary across single cells. While DNA and RNA are informational molecules, proteins compose the machines that carry out cellular processes. Thus, understanding single cell function requires highly sensitive measurements of protein abundance, interactions, and modification states; the technique ideally poised to provide such measurements is mass spectrometry-based proteomics. However, due to the absence of protein amplification methods, proteomic analysis of single cells can only analyze tens to hundreds of cells per day and focuses on processing each cell individually, similar to early single-cell nucleic acid technologies. Such methods are laborious, produce small amounts of material, and scale linearly. These challenges are particularly problematic because single-cell DNA/RNA sequencing has demonstrated that one must measure thousands of single cells to capture the cell-to-cell variation that exists, even amongst genetically identical cells, with many more required to analyze an organoid or tissue.

The single-cell revolution began with genomics and transcriptomics but remains incomplete. A need remains which allows for the expansion of single-cell analysis to proteins, and which can provide an easily accessible platform for understanding how these biochemical workhorses act in single cells.

### SUMMARY DISCLOSURE

In a first aspect, the disclosure provides a functionalized label, comprising a peptide of 3-20 amino acids in length, wherein the peptide is covalently bound to (a) one or more azide molecules, (b) one or more alkyne molecules, or (c) one or more azide molecules and one or more alkyne molecules.

In a second aspect, the disclosure provides a labeled protein comprising a first functionalized label according to the first aspect of the disclosure, covalently bound to the protein, wherein the first functionalized label comprises a first peptide.

In a third aspect, the disclosure provides a method of labeling a protein comprising: (a) contacting a protein with a first functionalized label to produce a single labeled protein, wherein the first the functionalized label comprises a functionalized label of the first aspect of the disclosure, wherein the peptide comprises a first peptide; and (b) contacting the single labeled protein with a second functionalized label to produce a double labeled protein, wherein the first the functionalized label covalently binds to the second functionalized label, and wherein the second functionalized label comprises a functionalized label of the first aspect of the disclosure, wherein the peptide comprises a second peptide.

In a fourth aspect, the disclosure provides a method comprising: (a) dividing a biological sample, comprising cells comprising individual proteins, into a plurality of separate first samples; (b) contacting each separate first sample with a different first functionalized label according to the first aspect of the disclosure, wherein each different first functionalized label comprises a different first peptide, wherein the contacting is carried out under conditions wherein each first functionalized label covalently binds to an individual protein in a plurality of proteins in the first sample that it is contacted with; (c) combining the plurality of separate first samples to generate a combined sample; (d) dividing the combined sample into a plurality of separate second samples; (e) contacting each separate second sample with a different second functionalized label according to the first aspect of the disclosure, wherein each different second functionalized label comprises a different second peptide, wherein the contacting is carried out under conditions wherein each second functionalized label covalently binds to the first functionalized label on the individual protein in the plurality of proteins in the second sample that it is contacted with; to produce a labeled plurality of proteins in a labeled biological sample; wherein the labeled plurality of proteins comprises a plurality of individual proteins from each individual cell in the biological sample and wherein each plurality of individual proteins from each individual cell is bound to a unique sequence of first and second functionalized labels.

In a fifth aspect, the disclosure provides a method comprising: (a) dividing a biological sample into a plurality of separate first samples; (b) contacting each separate first sample with a different first label, wherein each of the first labels binds to a plurality of proteins in each of the plurality of separate first samples; (c) combining the plurality of separate first samples to generate a combined sample; (d) dividing the combined sample into a plurality of separate second samples; (e) contacting each separate second sample with a different second label, wherein each of the second labels binds to the first label on the plurality of proteins in each of the plurality of separate second samples; and wherein the plurality of proteins from each individual cell in a biological sample is bound to a unique sequence of first and second labels.

### BRIEF DESCRIPTION OF FIGURES

FIG. 1 shows a diagram example for using split pool proteomics of the disclosure. (A) shows the methods of the disclosure. (B) shows the functionalized labels, labeled proteins and methods of labeling proteins of the disclosure. (C) shows exemplary results for the split pool proteomics of the disclosure.

FIG. 2 shows a diagram example of the functionalized label, labeled protein and method of labeling a protein of the first, second, third, fourth, and/or fifth aspects of the disclosure.

FIG. 3 shows a further overview of multiplexed combinatorial indexing. Cultured cells, organoids, or tissues are dissociated, fixed and distributed across a multiwall plate. Primary barcodes are affixed to proteins followed by re-pooling of cells, re-aliquoting, and affixing the secondary barcode directly to the primary barcode.

FIG. 4 shows a further examples of MMP combinatorial indexing incorporating two rounds of peptide-based barcoding.

FIG. 5 shows (A) workflow development for the development and application of Massively Multiplexed Proteomics (MMP). Novel chemical barcodes enable multiplexing of thousands of samples with cheap starting materials. Proteomes can then be quantified with standard instrumentation. (B) The total number of samples that can be multiplexed with MMP barcodes.

FIG. 6 shows (A) General chemoproteomic workflow for the identification of cysteines. Structures of cysteine labeling and capture shown in inset. (B) Fragmentation of click chemistry labeled peptides to release fragment ions and peptides. MMP harnesses this chemistry to release (C) shows a schematic for addition of primary and secondary barcodes.

FIG. 7 shows two-stage MMP peptide barcoding development. (A) Stage-1: Cysteines subjected to rounds of capping with iodoacetamide alkyne followed by cysteine-peptide-azide and termination with biotin, phosphonate, or a fluorophore. (B) Stage-2: Chemical simplification to two reactions using learned chemical utility from Stage-1 development to label with cysteine-reactive barcodes followed by click to alkyne barcode.

FIG. 8 shows extendable, fluorescent, and enrichable capping reagents for Stage-1 MMP barcodes (A) and (B).

FIG. 9 shows stage-2 barcode synthesis and validation. (A) Cysteines can be reacted with either a primary barcode or iodoacetamide alkyne to approximate combinatorial indexing. (B) Two barcodes have been synthesized that include biotin, an acid cleavable dialkoxydiphenyl linker, azide enrichment handle and pentapeptide (SEQ ID NO: 1 & 2). (C) Mass spectra of a sample prepared with click conjugation (SEQ ID NO: 3) to iodoacetamide alkyne labeled cysteines and 1:1 mixture of the GLMVA (SEQ ID NO: 1) and GLAVM (SEQ ID NO: 2) barcodes highlighting barcode-specific sequencing ions. (D) shows the synthetic scheme for linking the Stage 2 barcodes with a biotin affinity handle, DADPS linker, and an azide affinity handle and (E) that barcodes will be maintained as isobaric units to enhance analytical sensitivity.

FIG. 10 shows synthetic routes for two custom barcodes already synthesized for stage 2 barcoding scheme (iodoacetamide and DADPS).

FIG. 11 shows structures of iodoacetamide alkyne barcodes synthesized.

FIG. 12 shows structures of DADPS barcodes synthesized.

FIG. 13 shows the strategy for two rounds of sequential barcoding using iodoacetamide and DADPS barcodes.

FIG. 14 (A) shows structures of reporter ions detected for chemoproteomic analysis of NBIV-142-labeled samples (B) shows structures of reporter ions detected for chemoproteomic analysis of NBIV-143-labeled samples (C) shows (SEQ ID NO: 4), (D) shows representive MS/MS spectra labeled NBIV-124 and NBIV-143 (SEQ ID NO: 5), (E) shows representive MS/MS spectra labeled NBIV-124 and NBIV-143 (SEQ ID NO: 6), (F) shows representive MS/MS spectra labeled NBIV-124 and NBIV-143 (SEQ ID NO: 7).

FIG. 15 shows representative characteristic ion analysis reveals barcode specific ions (A) shows structures of NBIV-142 and NBIV-143 and frequency and intensity of identified characteristic ions for modified and unmodified peptide MS/MS spectra, (B) shows and frequency and intensity of additional identified characteristic ions for modified and unmodified peptide MS/MS spectra, (C) shows and frequency and intensity of additional identified characteristic ions for modified and unmodified peptide MS/MS spectra (D) shows structures and yields for prototype iodoacetamide-barcodes synthesized (from top to bottom SEQ ID NOs: 10-16), (E) shows structures and yields for additional prototype barcodes synthesized that include abiotic amino acids F-Fluoro substituent (from top to bottom SEQ ID NOs: 17-20).

FIG. 16 shows (A) Protein labeling with MMP-F. (B) MMP-F versus unlabeled cells. (C) MMP-F signal throughout cells. (D) MMP-F barcoded HCT116 cells with AF-555 or AF-647 mixed at a 1:1 ratio.

FIG. 17 shows a wide field of view of the in situ iodoacetemide-alkyne labeling of cysteines and read out by click azide-AF647.

FIG. 18 shows individual cellular in situ iodoacetemide-alkyne labeling of cysteines and read out by click azide-AF647.

FIG. 19. Mass spectrometric detection of barcoded peptides from whole cell proteomes. (A) Sequence for an example modified peptide (SEQ ID NO: 8). The modified peptide residue is highlighted in red. (B) Table of spectral matching features. B- and y-ions that were identified. The ppm mass errors for this high-resolution spectrum are shown for each ion. (C) Spectrum of the modified peptide from A. Matched b- and y-ions are annotated. Site determining fragment ions (b3/b4) are highlighted with the mass shift for the barcode peptide used here (SLGTC (SEQ ID NO: 21)). The spectrum was collected from a high-resolution analysis in an Orbitrap.

FIG. 20. Mass spectrometric detection of barcoded peptides from whole cell proteomes. (A) Sequence for an example modified peptide (SEQ ID NO: 9). The modified peptide residue is highlighted. (B) Table of spectral matching features. B- and y-ions that were identified are highlighted. The ppm mass errors for this ion trap spectrum are shown for each ion. (C) Spectrum of the modified peptide from A. Matched band y-ions are annotated. Site determining fragment ions (b8/b9 and y5/y6) are highlighted with the mass shift for the barcode peptide used here (SLGTC (SEQ ID NO: 21)). The spectrum was collected from an ion trap analysis.

FIG. 21 shows the Mass spectrometric (MS) method for identification and decoding of MMP peptides and post hoc quantification of barcoded peptides. (A) MS method schema, (B) match barcode to peptides, (C) quantify mixed barcodes.

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, CA), “Guide to Protein Purification” in Methods in Enzymology (M. P. Deutsheer, ed., (1990) Academic Press, Inc.); PCR Protocols: A Guide to Methods and Applications (Innis, et al. 1990. Academic Press, San Diego, CA), Culture of Animal Cells: A Manual of Basic Technique, 2nd Ed. (R. I. Freshney. 1987. Liss, Inc. New York, NY), Gene Transfer and Expression Protocols, pp. 109-128, ed. E. J. Murray, The Humana Press Inc., Clifton, N.J.), and the Ambion 1998 Catalog (Ambion, Austin, TX).

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gin; Q), glycine (Gly; G), histidine (His; H), isoleucine (Ile; I), leucine (Leu; L), lysine (Lys, K), methionine (Met; M), phenylalanine (Phe; F), proline (Pro; P), serine (Ser; S), threonine (Thr; T), tryptophan (Trp; W), tyrosine (Tyr; Y), and valine (Val; V).

As used herein, the singular forms “a”, “an” and “the” include plural referents unless the context clearly dictates otherwise.

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Additionally, the words “herein,” “above,” and “below” and words of similar import, when used in this application, shall refer to this application as a whole and not to any particular portions of the application.

In a first aspect, the disclosure provides a functionalized label, comprising a peptide of 3-20 amino acids in length, wherein the peptide is covalently bound to (a) one or more azide molecules, (b) one or more alkyne molecules, or (c) one or more azide molecules and one or more alkyne molecules.

As used herein, an azide molecule is a compound comprising the molecular formula N₃⁻. Azides are compatible with click chemistry procedures to add other functional moieties.

As used herein, an alkyne molecule is a compound comprising the molecular formula C₂. In one non-limiting embodiment, the alkyne is a cyclooctyne and comprises a molecular formula C₈H₁₂. Alkynes are compatible with click chemistry procedures to add other functional moieties. In various non-limiting embodiments the one or more alkyne molecules can include iodoacteamide alkyne, cycloalkyne, cyclooctyne, iodoacetamide alkyne, or alkyne NHS ester.

In various non-limiting embodiments the one or more alkyne molecules can also comprises a cysteine-capping alkyne molecule. The cysteine-capping alkyne molecule can be any cysteine-capping alkyne molecule suitable for use according to the disclosure. Examples of cysteine-capping alkyne molecule include, but are not limited to biotin, desthiobiotin iodoacetamide (DBIA), iodoacetimide, cysteine-reactive phosphate tags (CPT), N-ethylmaleimide (NEM), chloroacetamide, sulfonyl substituted heterocycles, iodoacetamide alkyne, or an azide.

In various embodiments, the functionalize label can comprise a peptide bound to one or more azide molecules; a peptide bound to one or more alkyne molecules; or a peptide bound to both one or more azide molecules and one or more alkyne molecules. According to these embodiments the one or more azide molecules and/or the one or more alkyne molecules can be bound to the peptide at the C-terminus; the N-terminus; both the C- and N-termini; or can be bound internally to an internal amino acid. In one non-limiting embodiment, a functionalize label comprising a peptide bound to an N- or C-terminal azide molecule, also comprises a C- or N-terminal cysteine as one of the 3-20 amino acids. In this embodiment, the C- or N-terminal cysteine reacts with and covalently binds to a composition comprising a peptide bound to an N- or C-terminal alkyne. This binding is compatible with click-chemistry procedures. In various other non-limiting embodiments the functionalized label can comprise an internally bound azide molecule and/or a peptide comprising a non-terminal (internal) cysteine as one of the 3-20 amino acids.

As used herein “click chemistry” is a class of biocompatible small molecule reactions commonly used in bio-conjugation, allowing the joining of substrates of choice with specific biomolecules.

As used herein the peptide in the functionalized label can comprise any peptide of 3-20 amino acids. In various non-limiting embodiments, the peptide is 3-19, 3-18, 3-17, 3-16, 3-15, 3-14, 3-13, 3-12, 3-11, or 3-10 amino acids in length, or wherein the peptide is 4-20, 4-19, 4-18, 4-17, 4-16, 4-15, 4-14, 4-13, 4-12, 4-11, 4-10, 5-20, 5-19, 5-18, 5-17, 5-16, 5-15, 5-14, 5-13, 5-12, 5-11, 5-10, 6-20, 6-19, 6-18, 6-17, 6-16, 6-15, 6-14, 6-13, 6-12, 6-11, or 6-10 amino acids in length.

In various embodiments the amino acids can include any naturally occurring amino acids and any non-naturally occurring amino acids. In one non-limiting embodiment, the peptide comprises the non-naturally occurring amino acid fluorine. In various other non-limiting embodiments the peptide comprises the amino acid cysteine as one of the 3-20 amino acids. In various examples of these embodiments the cysteine can be on the N-terminal of the peptide, the C-terminal of the peptide, or can be an internal amino acid.

According to the disclosure, the functionalized label can further include one or more functional moieties. As used herein a functional moiety or moieties are any molecule which can be used to purify, separate, enrich, detect, or visualize the functionalize label. These include, but are not limited to, probes or affinity tags. The one or more functional moieties can be any functional moiety suitable for use according to the disclosure. The affinity tag can be any affinity tag suitable for use according to the disclosure. Examples of affinity tags include, but are not limited to a streptavidin, hexahistidine tag, biotin, Desthiobiotin, a HA tag, a FLAG tag, or a phosphonate. The probes can be any probe suitable for use according to the disclosure and can include probes which are capable of visualization. Examples of probes capable of visualization include, but are not limited to florescent probes, fluorophores, isotopic labelling probes, or metal-based labelling probes. Examples of florescent probes include but are not limited to Alexa Fluor™ 647, 555, or 488. According to the disclosure, the one or more functional moieties can be bound to the peptide, or the one or more azide, or alkyne molecules. In one non-limiting embodiment the one or more functional moieties can be bound to the peptide via NHS esters.

According to the disclosure, the functionalized label can further include a cleavable linker. The cleavable linker can be any cleavable linker suitable for use according to the disclosure. A non-limiting example of a linker includes dialkoxydiphenylsilane (DADPS). In various embodiments the cleavable linker can be located anywhere in the functionalized label including, but not limited to, between the peptide and the one or more azide or alkyne molecules, between the peptide and a capping alkyne molecule, between the peptide and the one or more functional moieties, between the one or more azide or alkyne molecules and a capping alkyne molecule, or the one or more azide or alkyne molecules and the one or more functional moieties. The inclusion of a cleavable linker allows for the fragmentation and removal of parts of the functionalized label including, but not limited to the removal of a capping alkyne molecule, a functional moiety, or the removal of the azide or alkyne molecules.

In one non-limiting embodiment the functionalized label of the disclosure comprises (a) a peptide of 3-20 amino acids in length; (b) a branched azide molecule covalently linked to a caboxy terminus of the peptide; (c) a cleavable linker covalently linked to the branched azide molecule; (d) a biotin moiety covalently linked to the cleavable linker; and (e) an alkyne molecule covalently linked to the biotin moiety. In various non-limiting examples of the embodiment, the elements (b), (c), and (d) can be arranged and bound to the peptide (a) in any order and can be bound on the N- or C-termini of the peptide.

In a second aspect, the disclosure provides a labeled protein comprising a first functionalized label according to the first aspect of the disclosure, covalently bound to the protein, wherein the first functionalized label comprises a first peptide.

As used herein the “labeled protein,” “protein” to be labeled, or “individual protein(s)” can be any protein or string of polypeptides suitable for labeling and having at least 3 amino acids. In various non-limiting embodiments, the protein can be a short polypeptide of 3-100 amino acids in length. In various other non-limiting embodiments, the protein can be a larger protein of 50-10,000 amino acids in length. The size and amino acid sequence of the protein is not limited.

In another embodiment, this labeled protein can further comprise a second functionalized label according to the first aspect of the disclosure, wherein the second functionalized label comprises a second peptide. In this embodiment the second functionalized label is covalently bound to the first functionalized label or directly the protein so that the labeled protein now comprises two functionalized labels, a first functionalized label bound to the protein and a second functionalized label bound to the first label or directly to the protein.

In various further embodiments, the labeled protein with the first and second functionalized labels, can further comprise one or more additional functionalized labels according to the first aspect of the disclosure. In these embodiments, each additional functionalized label comprises an additional peptide. In these embodiments each additional functionalized label is covalently bound to a functionalized label present in the composition or directly to the protein. In one non-limiting example, the functionalized labels are added and bound to each other in serial, so that the first functionalized label is bound to the protein, the second functionalized label is bound to the first functionalized label, and each additional functionalized label is bound to the previously bound label, thereby creating a string of bound labels.

In one non-limiting embodiment, the first functionalized label is covalently bound directly to the protein via binding of one or more alkyne molecule in the first functionalize label to a cysteine residue in the protein. In another non-limiting embodiment, the first functionalized label is covalently bound to an iodoacetamide molecule which is directly covalently bound to a cysteine residue in the protein.

According to the disclosure, the covalent binding of the functionalized labels to each other can occur via binding of one or more azide molecule on one functionalized label to one or more alkyne molecule on another functionalized label.

In various non-limiting embodiments, the first peptide, the second peptide, and the one or more additional peptide in the functionalized labels all have a different amino acid sequences. In various other non-limiting embodiments, the first peptide, the second peptide, and the one or more additional peptides all have the same amino acid sequences.

The first functionalized label, the second functionalized label, and the one or more additional functionalized labels can be isobaric. Similarly the first peptide, the second peptide, and the one or more additional peptides can be isobaric. As used herein isobaric peptides or functionalized labels have the same or similar mass.

In a third aspect, the disclosure provides a method of labeling a protein comprising: (a) contacting a protein with a first functionalized label to produce a single labeled protein, wherein the first the functionalized label comprises a functionalized label of the first aspect of the disclosure, wherein the peptide comprises a first peptide; and (b) contacting the single labeled protein with a second functionalized label to produce a double labeled protein, wherein the first the functionalized label covalently binds to the second functionalized label, and wherein the second functionalized label comprises a functionalized label of the first aspect of the disclosure, wherein the peptide comprises a second peptide.

In one non-limiting embodiment, the method of labeling a protein comprising: (a) contacting the protein with the functionalized label of the first aspect of the disclosure, to produce a single labeled protein according to the second aspect of the disclosure; and (b) contacting the single labeled protein with the functionalized label of the first aspect of the disclosure, to produce a double labeled protein according to the second aspect of the disclosure.

In various non-limiting embodiments, the method can further comprise (c) contacting the double labeled protein with a thiol capping reagent to produce a thiol-capped double labeled protein. A thiol capping reagent can be any thiol capping reagent suitable for use according to the disclosure. Examples of thiol capping agents include, but are not limited to, biotin, desthiobiotin iodoacetamide (DBIA), iodoacetimide, cysteine-reactive phosphate tags (CPT), N-ethylmaleimide (NEM), chloroacetamide, sulfonyl substituted heterocycles, iodoacetamide alkyne, or an azide.

In other non-limiting embodiments, the method comprises repeating step (b), wherein each repeat of step (b) results in the binding of an additional functionalized label to the functionalized label previously bound to the protein or directly to the protein itself, and wherein each additional functionalized label comprises an additional peptide.

The methods according to the disclosure can comprise contacting the protein with a reducing agent prior to step (a). In this embodiment the reducing agent exposes thiols in one or more cysteines prior to contacting the protein with the functionalized label of the first aspect of the disclosure.

The methods according to the disclosure can also comprise contacting the protein with an iodoacteamide alkyne molecule prior to step (a). In this embodiment the iodoacteamide alkyne molecule covalently binds to the protein prior to contacting the protein with the functionalized label of the first aspect of the disclosure.

In various non-limiting embodiments the contacting the protein comprises contacting a biological sample comprising the protein.

As used herein the biological sample can be any kind sample suitable for use according to the methods, including but not limited to cells, organoids, and tissue sections. In non-limiting embodiments, the cells can be from cell cultures and the tissue sections can be isolated from whole organisms. In non-limiting embodiments, the biological sample can be chemically fixed and/or permeabilized.

In various non-limiting embodiments, the method comprises dividing the biological sample into a plurality of separate first samples prior to step (a). The method according to these embodiments can further comprise contacting each separate first sample with a different first functionalized label. The method according to these embodiments can further comprise combining the plurality of separate first samples to generate a combined sample and then dividing the combined sample into a plurality of separate second samples. The method according to these embodiments can further comprise contacting each separate second sample with a different second functionalized label.

In a fourth aspect, the disclosure provides a method comprising: (a) dividing a biological sample, comprising cells comprising individual proteins, into a plurality of separate first samples; (b) contacting each separate first sample with a different first functionalized label according to the first aspect of the disclosure, wherein each different first functionalized label comprises a different first peptide, wherein the contacting is carried out under conditions wherein each first functionalized label covalently binds to an individual protein in a plurality of proteins in the first sample that it is contacted with; (c) combining the plurality of separate first samples to generate a combined sample; (d) dividing the combined sample into a plurality of separate second samples; (e) contacting each separate second sample with a different second functionalized label according to the first aspect of the disclosure, wherein each different second functionalized label comprises a different second peptide, wherein the contacting is carried out under conditions wherein each second functionalized label covalently binds to the first functionalized label on the individual protein in the plurality of proteins in the second sample that it is contacted with; to produce a labeled plurality of proteins in a labeled biological sample; wherein the labeled plurality of proteins comprises a plurality of individual proteins from each individual cell in the biological sample and wherein each plurality of individual proteins from each individual cell is bound to a unique sequence of first and second functionalized labels.

According to this aspect of the disclosure, the method can also comprise repeating steps (a)-(e) with a one or more additional different functionalized labels according to the first aspect of the disclosure, wherein each repeat of steps (c)-(e) results in the binding of an additional different functionalized label to the functionalized label previously bound to the individual protein, and wherein each additional functionalized label comprises a different additional peptide.

According to this aspect of the invention a biological sample, which includes cells having a plurality of individual proteins, is divided into a plurality of separate first samples. The biological sample can be divided according to any method suitable for dividing up a biological sample into a plurality of separate first samples. According to this aspect each of the plurality of separate first samples comprises cells, and each cell comprises a plurality of individual proteins so that each separate first sample comprises a plurality of individual proteins. Each separate first sample (from the plurality of first samples) are then contacted with a different first functionalized label. Each different functionalized label includes a different peptide so that each separate first sample gets a different first functionalize label with a different first peptide. Each different first functionalized label can also have one or more different azide and alkyne molecules and/or one or more different functional moieties. The contacting is carried out under conditions wherein each first functionalized label covalently binds to the individual proteins in a plurality of proteins in each first sample that it is contacted with the first functionalized label. The plurality of separate first samples which comprise individual proteins covalently bound to different first functionalized labels are then combined back together to generate a combined sample. The combined sample is then divided a second time into a plurality of separate second samples. So that each separate second sample comprises a plurality of individual proteins covalently bound to a first functionalized label. Each separate second sample (from the plurality of second samples) are then contacted with a different second functionalized label. Each different functionalized second label includes a different peptide. Each different second functionalized label can also have one or more different azide and alkyne molecules and/or one or more different functional moieties. The contacting is carried out under conditions wherein each second functionalized label covalently binds to the first functionalized label, which is already covalently bound the individual proteins which make up in the plurality of proteins in the plurality of separate second samples.

These steps can be repeated multiple times in order to combine each separate sample to produce a combined sample and then divide the combined sample into a plurality of different samples. Additional different functionalized labels can then be covalently bound to the functionalized label previously bound to the individual protein, and wherein each additional functionalized label comprises a different additional peptide. Each different functionalized label can also have one or more different azide and alkyne molecules and/or one or more different functional moieties.

The multiple dividing and contacting steps produce a labeled biological sample which comprises a labeled plurality of proteins. Each individual protein in the labeled plurality of proteins is covalently bound to a sequence of first and second (and optionally additional different) functionalized labels. Each plurality of individual proteins from each individual cell will be bound to a unique sequence of functionalized labels. Thus, individual proteins with the same unique sequence of functionalized labels can be determined to be from the same individual cell in the biological sample. This method allows for the unique labeling of the individual proteins from an individual cell in a biological sample.

In various non-limiting embodiments, the first peptide, the second peptide, and the one or more additional peptide in the functionalized labels all have a different amino acid sequences. In various other non-limiting embodiments, the first peptide, the second peptide, and the one or more additional peptides all have the same amino acid sequences.

The first functionalized label, the second functionalized label, and the one or more additional functionalized labels can be isobaric. Similarly the first peptide, the second peptide, and the one or more additional peptides can be isobaric. As used herein isobaric peptides or functionalized labels have the same or similar mass.

According to the methods of the disclosure, the functionalized labels bind to the majority of the plurality of proteins or the previously bound label during each of the contacting steps.

The methods of the invention can be repeated and additional labels added onto the end of the previously added label from the prior contacting step round. The repetition of additional rounds of label addition allows for the processing of larger amounts of sample. This method of pairing combining (pooling) and dividing (redistribution) of cells can be referred to as split-pooling.

In one non-limiting embodiment, the dividing and contacting steps are conducted using a multi-well plate. According to this embodiment, the biological samples is divided into a plurality of wells, wherein each well comprises a separate first sample; a different functionalize first label is added to each well. After covalent binding of the functionalized label to the proteins in the separate first sample, the separate samples are combined and then divided into a plurality of wells a second time, wherein each well comprises a separate second sample. A different second functionalize label is added to each well and the second functionalize label covalently binds to the first functionalize label which is bound to the individual proteins in the samples. These steps can be repeated to add additional different functionalize labels onto the previously bound functionalize label(s).

As used herein the biological sample can be any kind sample suitable for use according to the methods, including but not limited to organoids and tissue sections. In non-limiting embodiments, the cells can be from cell cultures and the tissue sections can be isolated from whole organisms.

In non-limiting embodiments, the biological sample can be chemically fixed and/or permeabilized.

According to the disclosure, the method can further comprise (f) contacting the labeled plurality of proteins with one or more thiol capping reagent. The thiol capping reagent can be any thiol capping agent as discussed in the first aspect of the disclosure. In various non-limiting embodiments, the thiol capping reagent can also comprise one or more functional moieties. The functional moiety can be any functional moiety as discussed in the first aspect of the disclosure.

According to the disclosure, the method can comprise detecting the different functionalized labels bound to the individual proteins and analyzing the different functionalized labels bound to the individual proteins.

According to the method of the disclosure, detecting can further comprise steps which denature, reduce, alkylate, digest, remove contaminants/salts, and/or fractionate the samples. Additional steps can include steps which assist in batch correction, control processing, and/or labeling efficiency calculations to confirm all functionalized labels are efficiently labeling proteins.

According to the disclosure, detecting and analyzing can comprise (g) lysing the cells in the labeled biological sample; (h) isolating the labeled plurality of proteins; and (i) identifying unique sequence of functionalized labels bound to individual proteins present in the labeled plurality of proteins. Identifying can also comprise identifying the order and/or quantity of each unique sequence of functionalized labels and/or each different functionalized label. Identifying can also comprise determining the amino acid sequence of each individual protein and/or each different functionalized label. Each of the individual proteins bound to the same unique sequence of functionalized labels indicates that the individual proteins are from the same individual cell in the biological sample.

Lysing the cells in the labeled biological sample can be accomplished using any suitable method including, but not limited to contacting with detergents, mechanical disruption, syringe pumping, liquid homogenization, high frequency sound waves (sonication), freeze/thaw cycles, and manual grinding.

Isolating the labeled plurality of proteins can be accomplished using any suitable method, including, but not limited to using functional moieties on the functionalized labels, chromatography (including high-performance liquid chromatography (HPLC) and/or thin-layer chromatography (TLC)), centrifugation, affinity capture, magnetic separation, and/or size exclusion chromatography.

Identifying the unique sequences of functionalized labels bound to individual proteins present in the labeled plurality of proteins can be accomplished using any suitable method, including, but not limited to using to using functional moieties on the functionalized labels, HPLC, mass spectrometry, chromatography, antibody-based detection assays, western-blotting, affinity purification, magnetic separation, and/or enzyme-linked immunosorbent assay (ELISA).

In various non-limiting embodiments, the detecting and analyzing further comprises digesting the labeled plurality of proteins. Digesting can be used to produce shorter labeled proteins, prior to the identifying step (i). According to these embodiments, the digesting can be accomplished using any suitable method, including, but not limited to, contacting with proteases, including trypsin, LysC, ArgC, GluC, chymotrypsin, protease K.

In various non-limiting embodiments, when the functionalized label comprises a functional moiety, the detecting and analyzing further comprises enriching the labeled plurality of proteins using the functional moiety. In various other non-limiting embodiments the labeled plurality of proteins can be enriched using centrifugation, antibody based enrichment, affinity based enrichment, or HPLC. In various other non-limiting embodiments, the labeled plurality of proteins is fractionated using reverse-phase chromatography.

A functionalized label comprising one or more functional moieties capable of visualization can be used for monitoring the effectiveness of the labeling and tracking of individual cells in the methods of the disclosure. In one non-limiting embodiments, a fluorescent microscope can be used to visualize the functional moiety.

According to the methods of the disclosure, the detecting and analyzing can further comprise quantifying the amount of each of the individual proteins, and/or the amount of each different functionalized label, and/or the amount of each unique sequence of functionalized labels. In various non-limiting embodiments the detecting and analyzing further comprises identifying each individual protein's and/or different functionalized label's amino acid sequence.

Identifying and/or quantifying can be accomplished using any suitable method, including, but not limited to, mass spectrometry, chromatography, western-blotting, and/or enzyme-linked immunosorbent assay (ELISA).

In various non-limiting embodiments quantifying the amount of each of the individual proteins and/or the amount of each different functionalized label is based on chromatographic elution profiles using chromatography, and/or profile intensities using mass spectrometry.

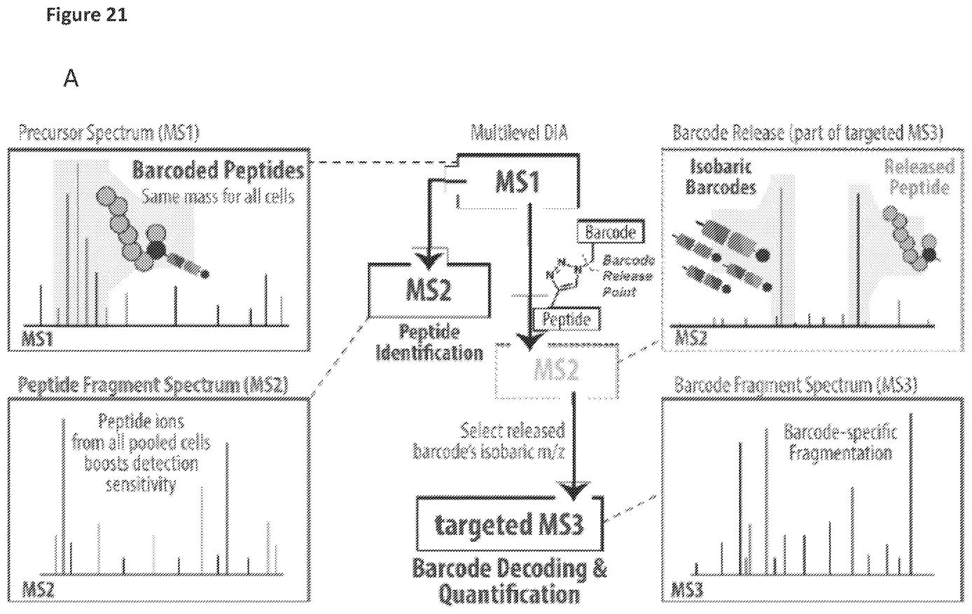

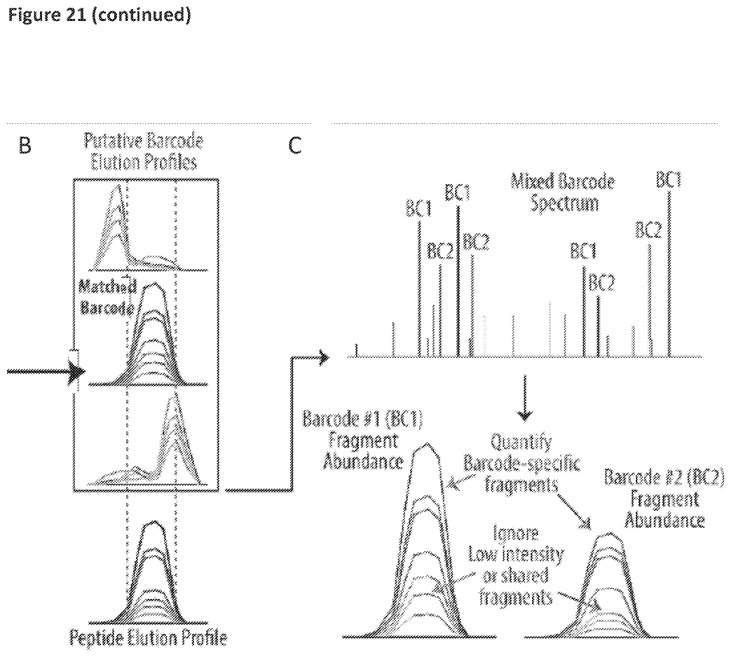

In one, non-limiting embodiment (as shows in FIG. 21), the identifying and/or quantifying comprises: (i) performing mass spectrometry on the labeled plurality of proteins to produce a precursor spectrum (MS1); (ii) fragmenting the different functionalized labels from the labeled plurality of proteins, wherein the fragmenting is carried out under conditions to break the covalent bond between the different functionalized labels and the individual proteins; (iii) performing mass spectrometry on the different functionalized labels to produce a functionalized labels spectrum (MS2) and quantifying an amount of each different functionalized label and/or each unique sequences of functionalized labels; (iv) performing mass spectrometry on the plurality of individual proteins to produce a protein spectrum (MS3), quantifying an amount of each individual protein, and identifying each individual protein; and (v) matching the functionalized labels spectrum (MS2 or higher) to the protein spectrum (MS2 or higher) to determine the quantity and identity of individual proteins from the same individual cell. In various non-limiting embodiments, the identification and quantification can be performed at MS2 or MS3 or additional at later mass spectrometry (MS) steps. In various other embodiments, the fragmenting step can also fragment the different functionalized labels from each other, thereby break the covalent bonds between the different functionalized labels.

As used herein analyzing can comprise performing any method which allows for the quantification of the amount of each unique sequence and identity of labels bound to the labeled plurality of proteins. Analyzing can also comprise performing any method which allows for the determination of the amino acid sequence of each protein and/or different functionalized label. Non-limiting embodiments can include any type of imaging, mass spectrometry, western blotting, flow cytometry, and/or Fluorescence-Activated Cell Sorting (FACS).

In one, non-limiting embodiment, the identifying and/or quantifying comprises the labeled plurality of proteins being separated by LC and analyzed by tandem mass spectrometry (LC-MS/MS). According to this embodiment, the labeled plurality of proteins are identified in MSI spectra and fragmented to release the functionalized labels. Depending on the label chemistry, this can be done at low energy to only release the label, if additional rounds of fragmentation and quantification are desired. Otherwise, the protein and label is fragmented simultaneously in the mass spectrometer. Functionalized label-specific peaks are identified and quantified based on ion intensities with or without supplemental quantitative tagging (e.g., TMTpro). Quantified proteins and labels are matched using canonical MS/MS database searching and a custom method for quantitation and deconvolution of labels (searching for specific ions for each label and quantifying the relative intensities of the release of these ions). The quantification is calibrated, as not all fragmentation events happen equivalently. Individual label quantitation is indicative of an individual cell assuming a high enough density of labels individual proteins.

In various embodiments, the biological sample is a mammalian cell sample.

In a fifth aspect, the disclosure provides a method comprising: (a) dividing a biological sample into a plurality of separate first samples; (b) contacting each separate first sample with a different first label, wherein each of the first labels binds to a plurality of proteins in each of the plurality of separate first samples; (c) combining the plurality of separate first samples to generate a combined sample; (d) dividing the combined sample into a plurality of separate second samples; (e) contacting each separate second sample with a different second label, wherein each of the second labels binds to the first label on the plurality of proteins in each of the plurality of separate second samples; and wherein the plurality of proteins from each individual cell in a biological sample is bound to a unique sequence of first and second labels.

As used herein the first label can comprise any small molecule which can bind to the plurality of proteins. Non-limiting examples include peptides, nucleotides (DNA, RNA, PNA), small organic molecules, heteropolymers, biopolymers, or small chemical molecules. The second and plurality of labels can comprise any small molecule which can bind to the first label. Non-limiting examples include peptides, nucleotides (DNA, RNA, PNA), small organic molecules, heteropolymers, biopolymers, or small chemical molecules.

In various non-limiting embodiments, the method can comprise repeating steps (a)-(e) with a plurality of labels, wherein each repeat of steps (c)-(e) results in the binding of an additional label to the label previously bound to the individual protein.

In one, non-limiting embodiment the label can be bi-functional and comprise more than one part.

According to the methods of the disclosure the labels bind to the majority of the plurality of proteins or the previously bound label during each of the contacting steps.

According to this embodiment, the labels can comprise acceptor chemistry as the first part and a unique small molecule as the second part. In this embodiment, the acceptor chemistry binds to the small molecule and the combination of the acceptor and small molecule make up each “label.” According to the methods of this embodiment, the endogenous amino acid(s) of the plurality of proteins can be targeted selectively with chemistry X to create sites for chemistry Y. Chemistry Y then attaches the unique small molecule label to the protein, which terminates in a site compatible with chemistry X, facilitating iterative rounds of additional label binding.

In other non-limiting embodiments, the labels can also serve other functions, including but not limited to increasing the solubility of the biological sample using an acid/base rich label; facilitating selective purification using an affinity tag (biotin, desthiobiotin); improving chromatographic retention; and/or improved ionization.

As used herein “acceptor chemistry” can be any chemistry which allows the addition of unique small molecule labels to a plurality of proteins. In various embodiments, this can include amine-reactive, C-terminal reactive, N-terminal reactive, and/or acid reactive chemistries, and/or cys reactive Michael addition reactions (which include, but are not limited to chloroacetamide and maleimide).

In a non-limiting embodiment, the acceptor chemistry comprises thiol-reactive components and azide-alkyne chemistry, and can be selective for cysteines in the plurality of proteins or previously bound peptide labels. According to the methods of this embodiment, iodoacemtemide reacts with endogenous cysteine residues in the plurality of proteins or the previously bound label; then copper catalyzes azide-alkyne cycloaddition. The two chemistries are bridged with the bi-functional label as follows: 1) iodoacetemide-alkyne 2) azide—unique peptide label—cysteine.

In a further non-limiting embodiment, selective labeling of particular amino acids can be used and is helpful in the deconvolution of the peptide sequences and labels.

In one non-limiting embodiment, the acceptor chemistry can be the same for all of the labels.

Within each individual contacting step, each separate sample is contacted by a different label. However, the same label could be used between rounds. In this non-limiting embodiment, the same label could be added to a separate sample in the first contacting step and the second contacting step. This embodiment would result in the plurality of proteins from an individual cell bound to a sequence of two copies of the same label.

### EXAMPLES

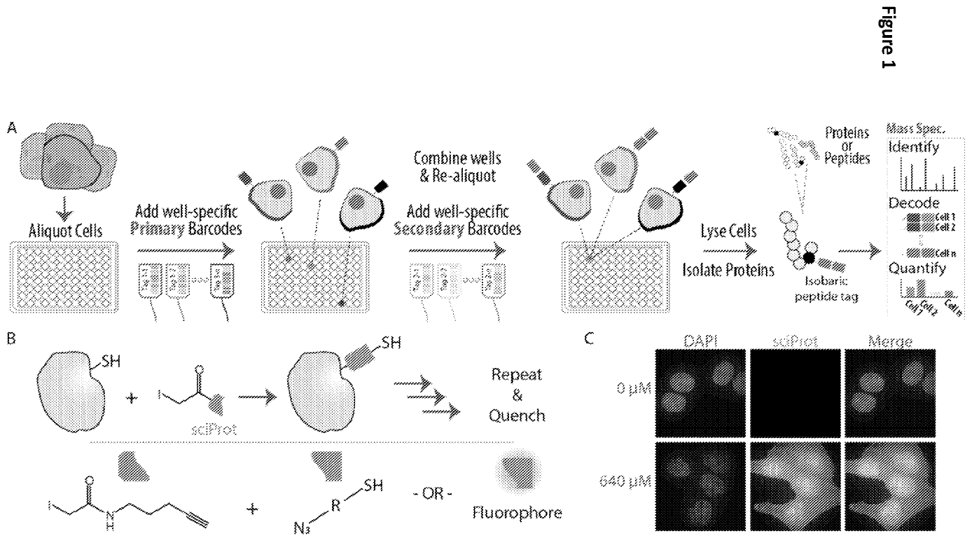

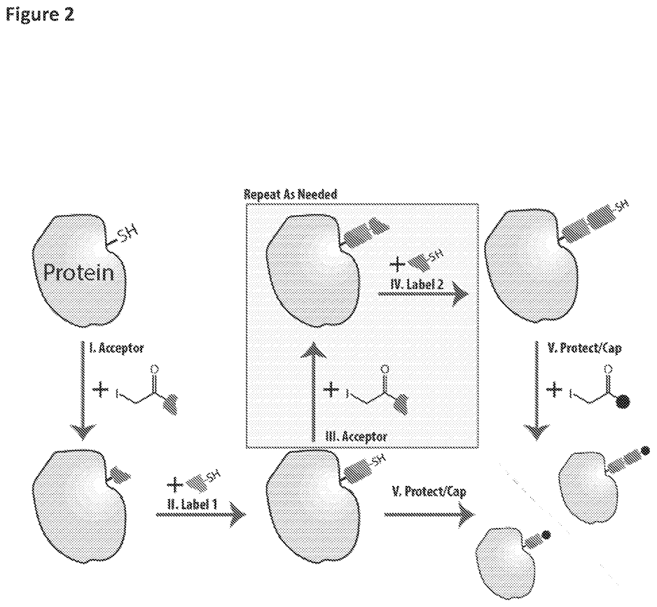

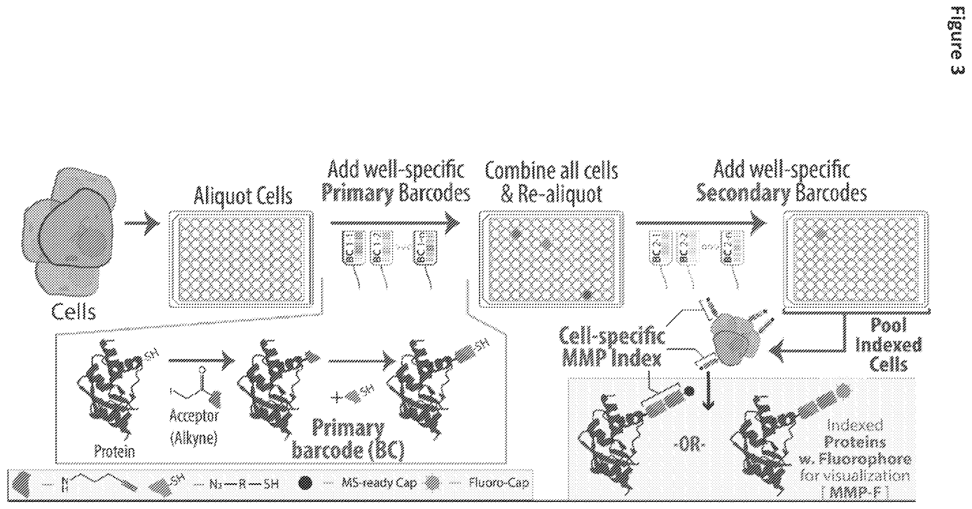

Functionalized labels and methods for Massively Multiplexed Proteomics (MMP) was developed which combines combinatorial indexing and peptide-based barcodes to achieve an exponential improvement in single-cell throughput using available proteomics workflows and instruments (FIGS. 1-5). By establishing the innovations of MMP, quantitative single-cell proteomics can now be as routine as single cell sequencing.

The major drawback with current methods was balancing cell throughput (the number of cells that can be processed per day) and proteome depth (the number of proteins that can be quantified per cell). To capture the diversity of cellular systems, single-cell proteomics required a paradigm shift to achieve coverage at the level of thousands of cellular proteomes per day.

The current methods pairs combinatorial indexing and peptide barcoding to achieve the technological leap needed for single cell proteomics. Combinatorial indexing pairs pooling and redistribution of cells (split-pooling) and multiple rounds of barcoding (combinatorial indexing). Combinatorial indexing bypasses requirements for complex and costly single-cell manipulation and therefore is ideally suited to generate low cost, and robust single cell genomics data. With combinatorial indexing, a population of cells was distributed at random across a multi-well plate, each well receiving many cells^{10,11}. With the cells kept intact, each well was affixed with a unique primary barcode. Cells were then pooled back together and redistributed for another round of barcoding. Combinatorial indexing can be applied on either intact cells or isolated nuclei.

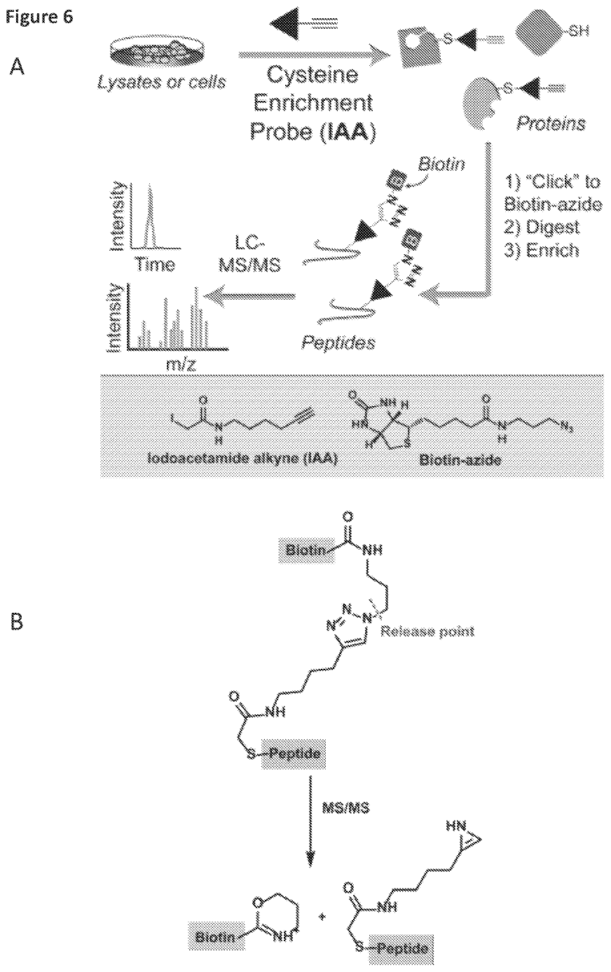

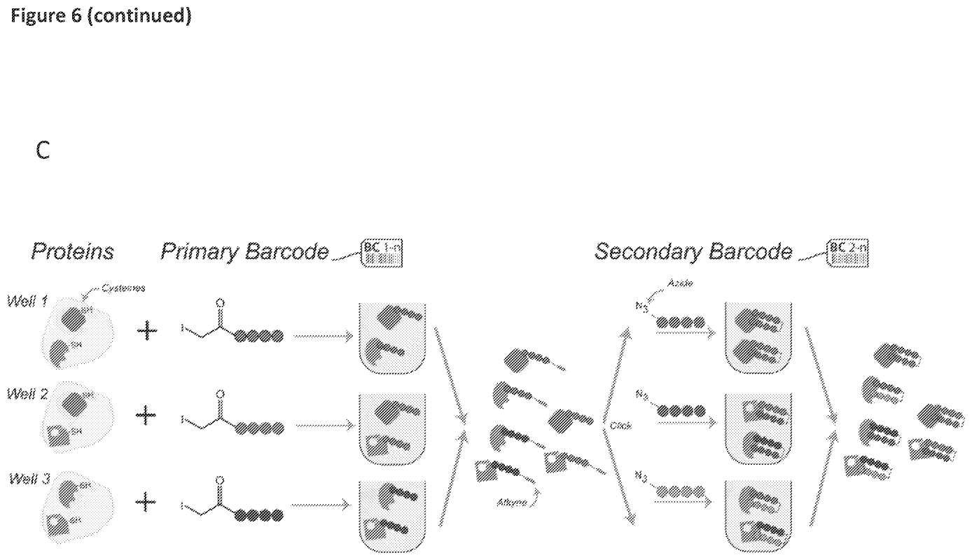

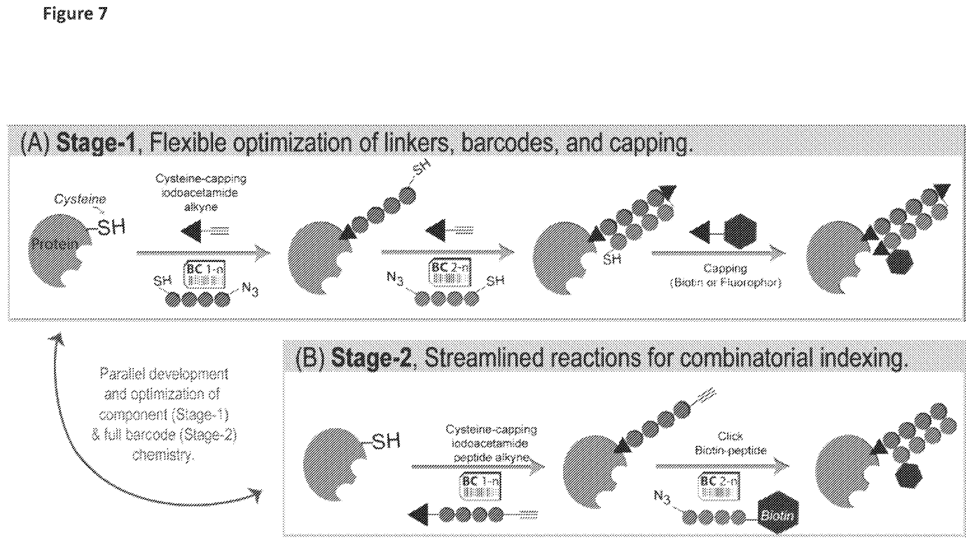

Cysteine-based labeling was used for incorporation of barcodes into samples; using pan-cysteine reactive reagents such as iodoacetamide alkyne clicked to biotin-azide (FIG. 6A). For alkyne-based probes, click chemistry was used for subsequent conjugation to biotin-based capture reagents. Subsequently, cysteine containing peptides were easily enriched after tryptic digestion.

Isobaric reagents were utilized to quantify cysteine-containing peptides for multiplexed cysteine chemoproteomics. During tandem mass spectrometry, reagents released isotopically encoded reporter ions that enable quantification of the relative abundance of peptides and proteins. To maintain an isobaric modification mass on peptides from each sample, an inversely encoded balancer region is released simultaneously with release of the reporter ions.

The result was that chemically identical peptides from different samples that are functionalized with different isobaric mass tags were indistinguishable as peptide precursor ions so that many samples (e.g., biological or technical replicates) could be combined and analyzed together without any increase in sample complexity. Upon tandem mass spectrometry, the abundance of a peptide in each sample was readily identified from the intensity of the released reporter ions. Despite the widespread utility of isobaric labeling, the current limit for sample multiplexing using this technology is 18 samples. To achieve the >500 sample multiplexing required for single-cell proteomics of cancer cells and tumors required the development of the instant new approach.

Unlike current proteomics multiplexing reagents that rely on a limited number of positions amenable to heavy isotope incorporation, MMP's peptide-based barcodes can achieve nearly unlimited sample multiplexing. Exemplifying the untapped opportunities, two rounds of barcoding each with simple four amino acid peptide sequences would afford a theoretical limit of 576-plex multiplexing. The pairing of cysteine bioconjugation with click chemistry is the ideal chemistry for protein-based combinatorial indexing and development of the instant MMP disclosure.

### Example 1

### Materials and Methods

Preparation of Functionalize labels. Peptides were manually synthesized according to the following general procedure.

Loading of the 2-chlorotrityl chloride resin: 2-chlorotrityl chloride resin (100-200 mesh, 0.1-0.9 mmol/g) was added to a solid-phase vessel and swelled in dry CH₂Cl₂ for 1 hr. The CH₂Cl was vacuum filtered off and first fmoc protected amino acid (2 Eq) was dissolved in dry CH₂Cl and diisopropylethylamine (DIPEA) (3Eq.) and loaded onto resin. This was left to incubate for 1 hr, after which the solution was vacuum filtered off and resin washed thoroughly with CH₂Cl, DMF, and MeOH. In the case of N-Fmoc-Biotin-Lys, the loading step was performed in DMF.

Amino Acid Coupling: Coupling of standard amino acids was carried out through treatment of the deprotected resin with 3 equivalents of Fmoc-protected amino acids, 3 equivalents of N,N,N′,N′-Tetramethyl-O-(1H-benzotriazol-1-yl) uronium hexafluorophosphate (HBTU), and 6 equivalents of DIPEA in DMF for 30 min. Each coupling was performed twice unless the amino acid used was valuable in which case the coupling was left longer. In between coupling and deprotection steps the resin was washed thoroughly with CH₂Cl₂, then DMF, MeOH, and CH₂Cl₂. Iodoacetic acid coupling was performed with propanephophonic anhydride (T3P) coupling reagent. Coupling of compounds to resin was monitored using the Kaiser test.

Fmoc Deprotection: Removal of N-terminal Fmoc protecting groups was carried out by treating the resin with 50% 4-methylpiperidine in DMF (3×1 min). After deprotection the resin was washed thoroughly with CH₂Cl, then DMF and CH₂Cl₂. Complete deprotection was monitored using the Kaiser test.

Resin Cleavage: Fully assembled peptides were then thoroughly washed with CH₂Cl and the dried resin was incubated with 20% hexafluoroisopropanol (HFIP) for 10 minutes. The resulting solution was collected into a round-bottom flask and the process was repeated two times. The collected peptide solution was concentrated down to 2-3 mL and precipitated into cold diethyl ether. The ether was decanted off and the peptide dissolved in water. The desired peptides were obtained as fluffy white-pale yellow solids after lyophilization. LC-MS analysis revealed only minor impurities and barcodes were used without further purification.

Labeling of cellular samples with thiol-reactive and click chemistry. Human HCT116 cells were fixed with 4% paraformaldehyde for 10 minutes and permeabilized with Trtion X-100. Permeabilized cells were reduced in TCEP and then aklyated with 2 mM iodoacetamide alkyne. After washing, an azide-conjugated Alexa Fluor dye was added at 5 μM in copper-cataylzed click buffer. The click reaction was washed, and the samples were mounted in anti-fade mounting media containing DAPI. Fluorescent imaging of labeled samples was performed using a spinning disc confocal microscope.

### Results

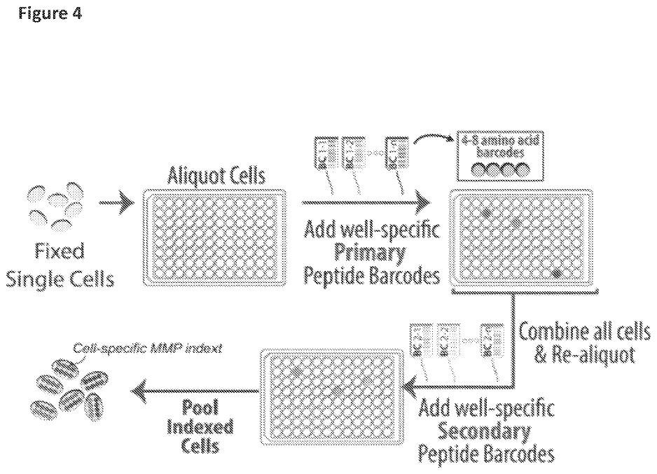

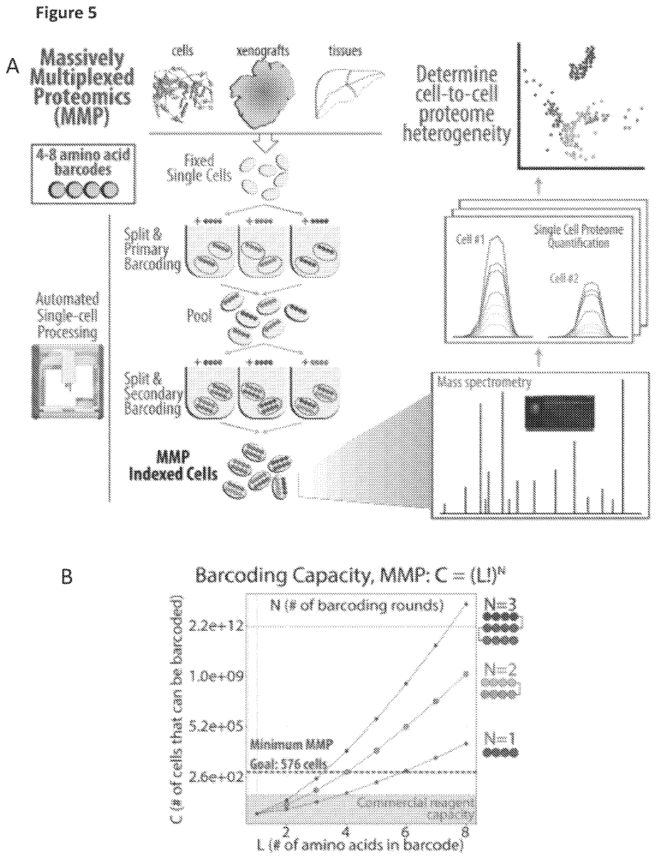

The proteins from individual cells were chemically encoded by a series of peptide-based molecular barcodes, creating a unique MMP cell index for the measurement of single-cell proteomes (FIG. 4).

Establishing the MMP barcoding chemistry. Peptides are ideally suited to serve as barcodes for MMP because they are high-density information-carrying units that are easily decoded by mass spectrometry-based proteomics. First cysteines were capped with the pan-cysteine reactive iodoacetamide alkyne acceptor reagent followed by click conjugation to azide- and thiol-containing peptide barcodes. The inclusion of the thiol in the barcode sequence allowed for sequential rounds of capping with iodoacetamide alkyne followed by click conjugation barcoding. Lastly, after rounds of barcoding were complete, the thiol moiety was capped with enrichment handles (e.g. biotin, desthiobiotin, or CPT), which capture peptides using immobilized metal affinity¹⁶ or streptavidin binding^{14,15}. The barcoded peptides were decoded based on the unique fragmentation of the triazole and thioether groups to release the barcodes from the labeled peptides, in a manner analogous to the fragmentation of gas phase cleavable crosslinkers (e.g., sulfoxide) routinely used in the study of protein interactions by mass spectrometry.

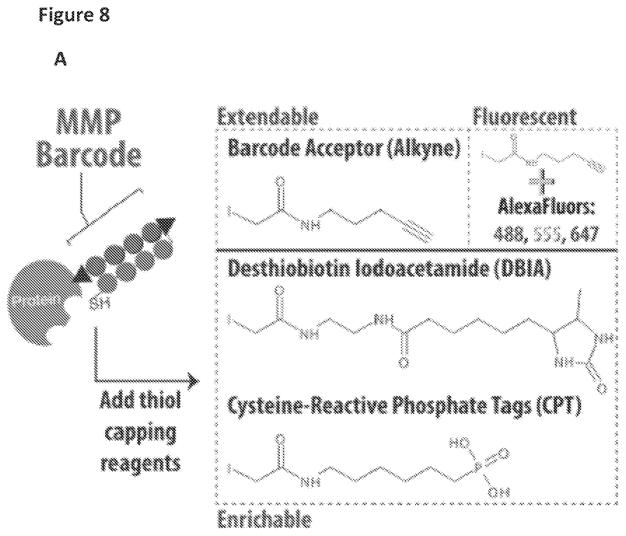

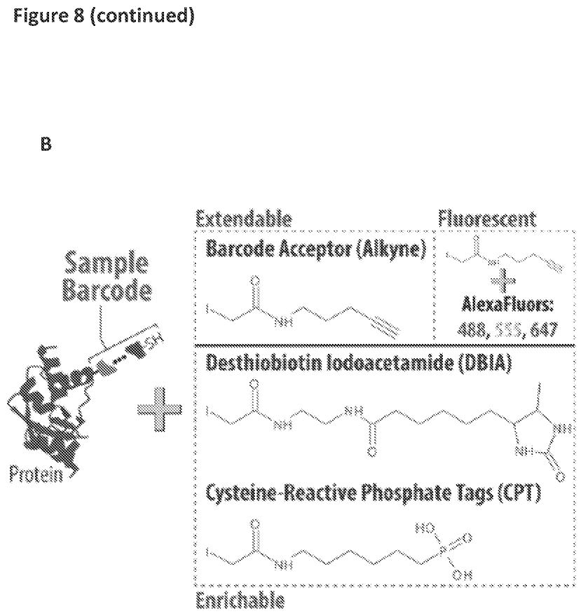

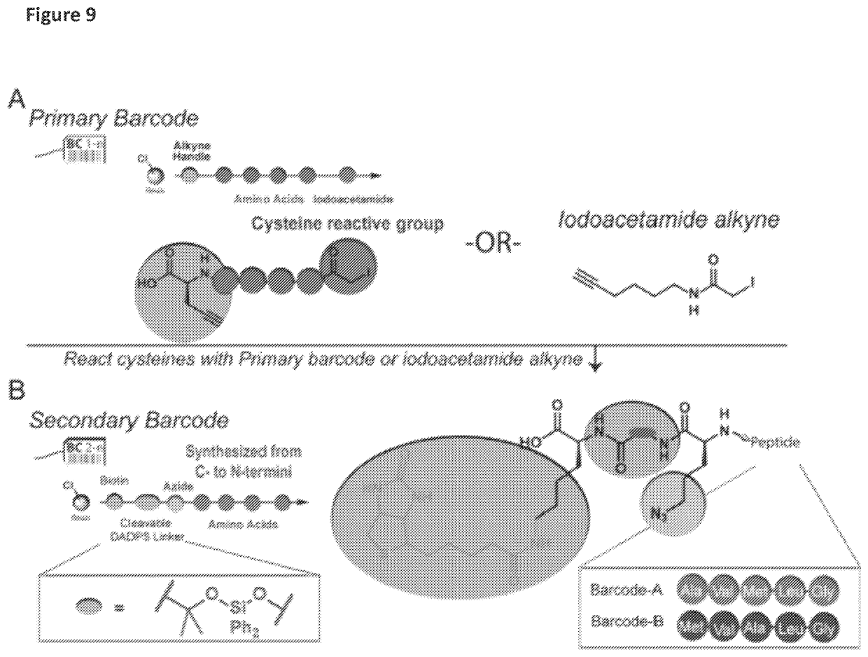

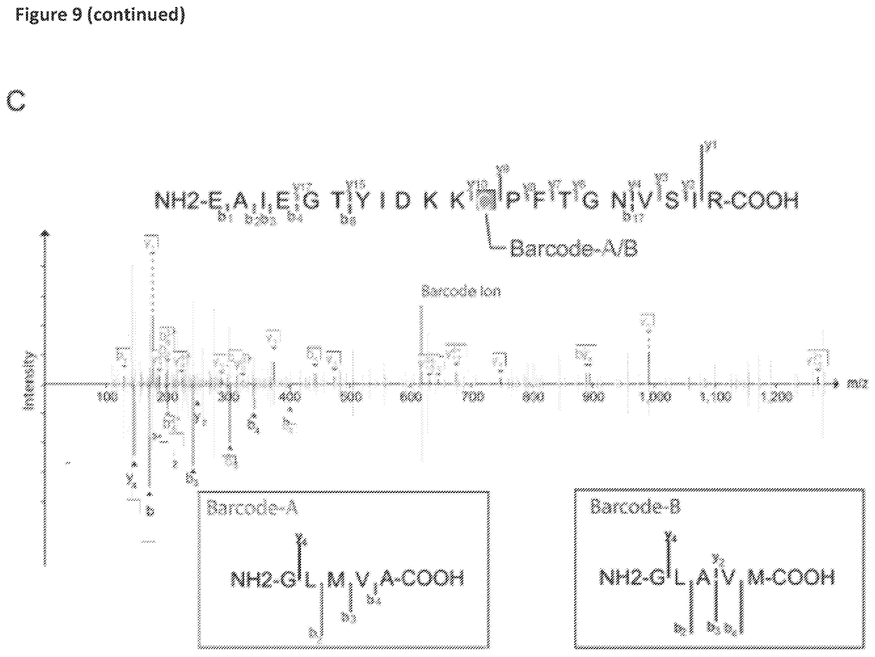

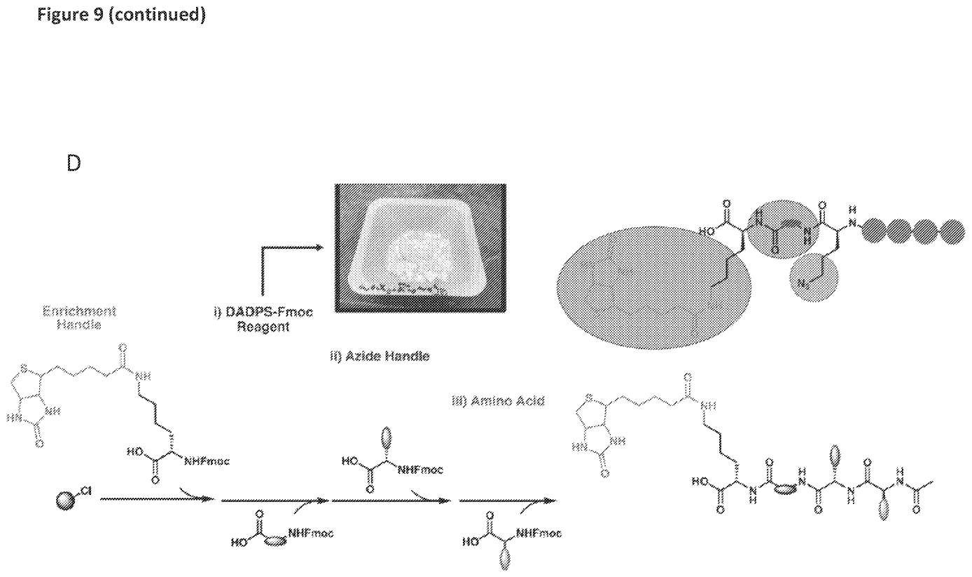

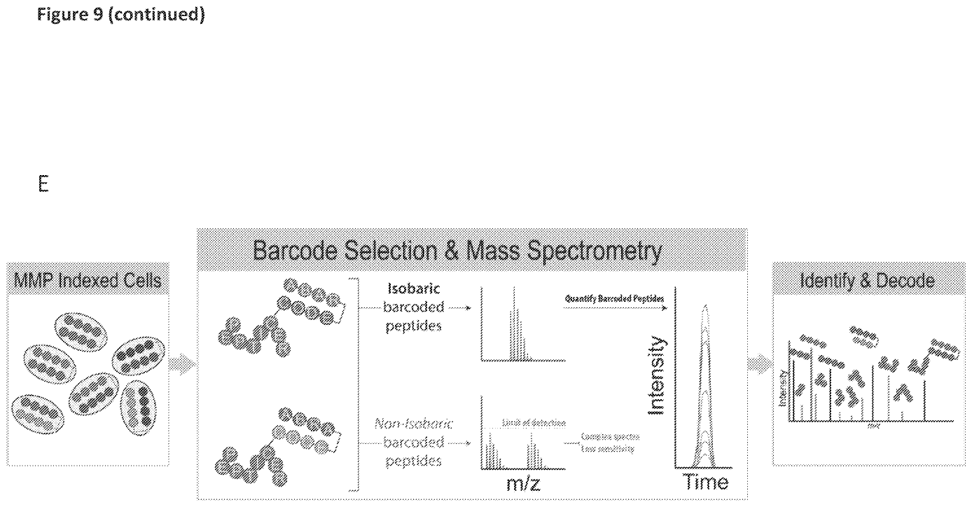

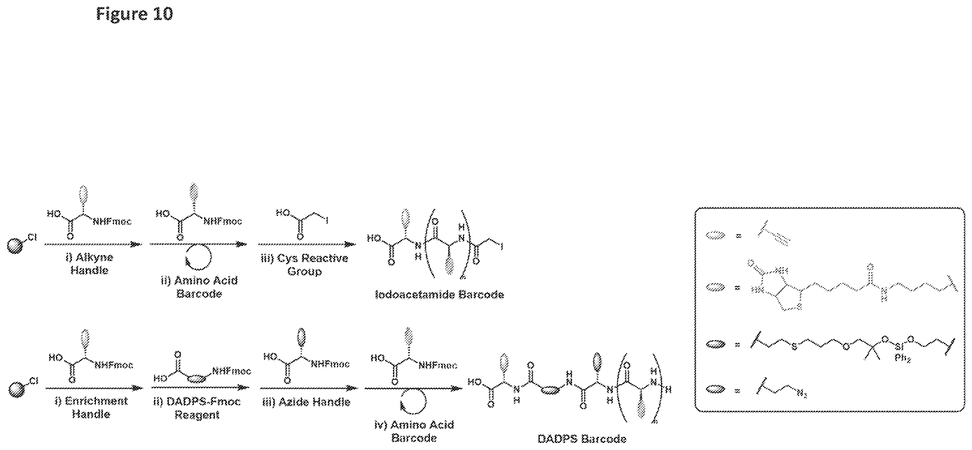

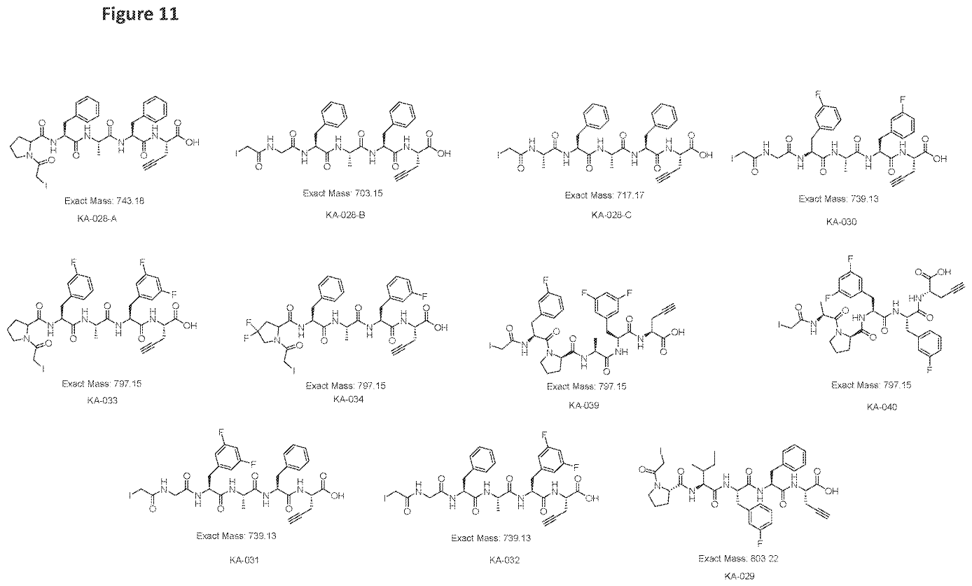

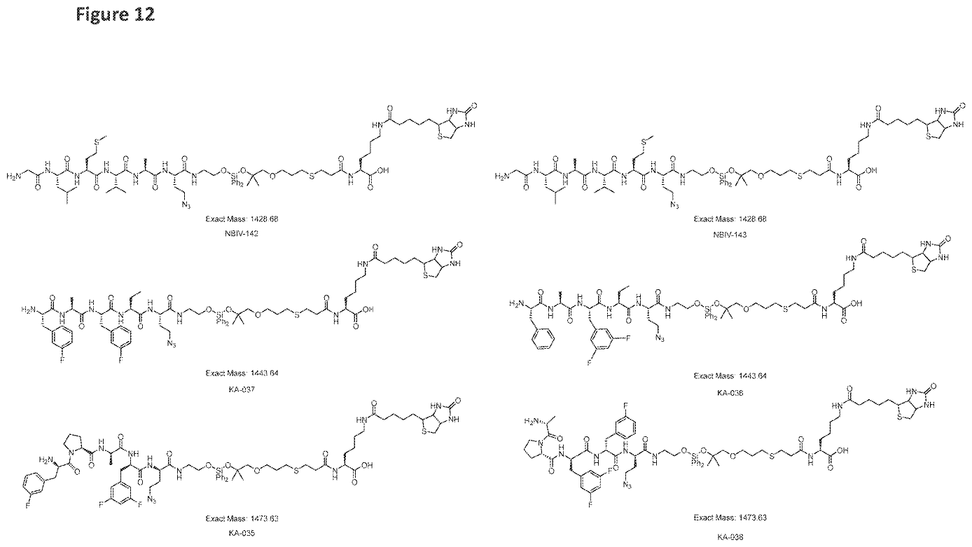

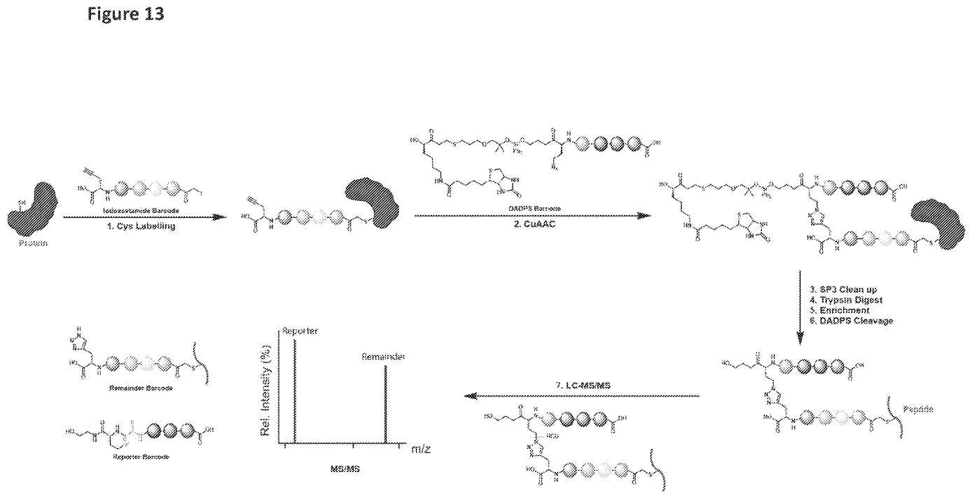

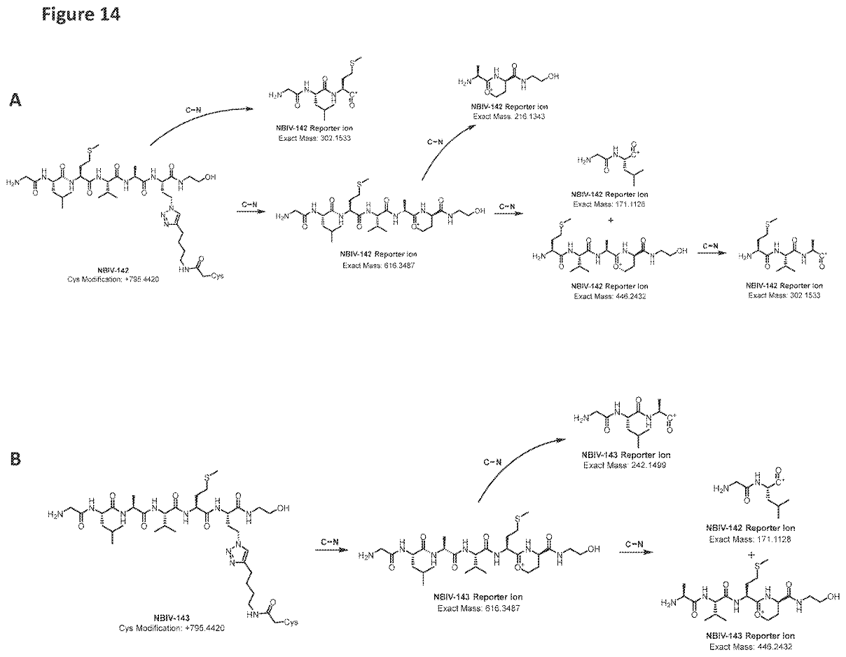

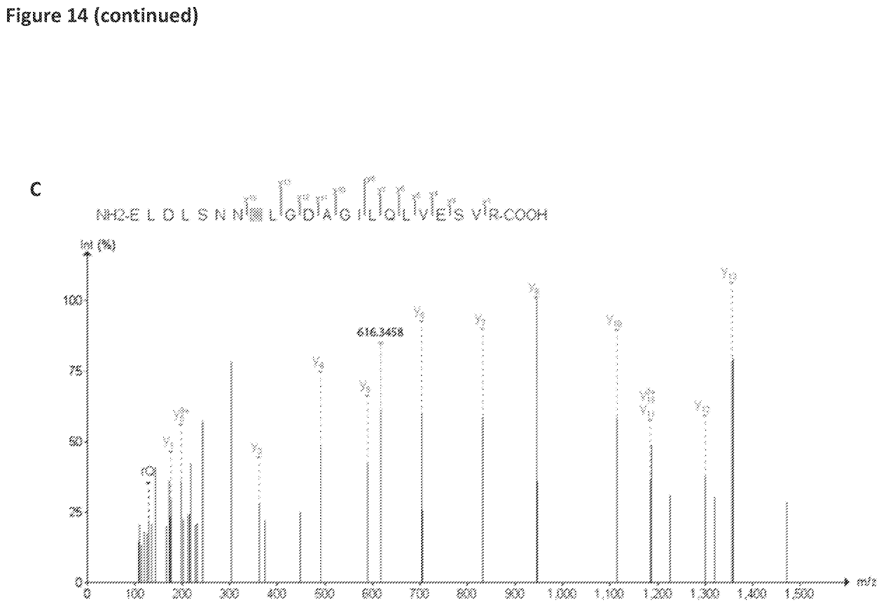

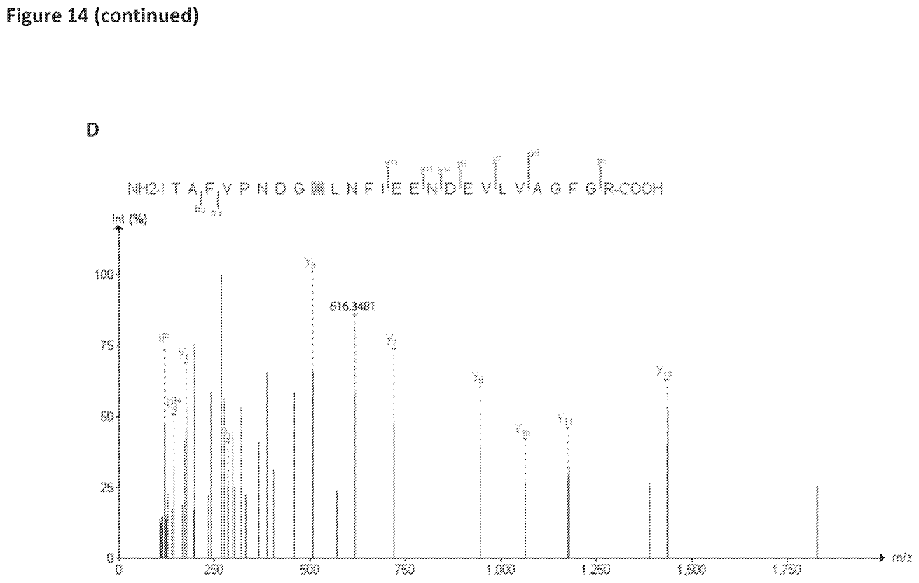

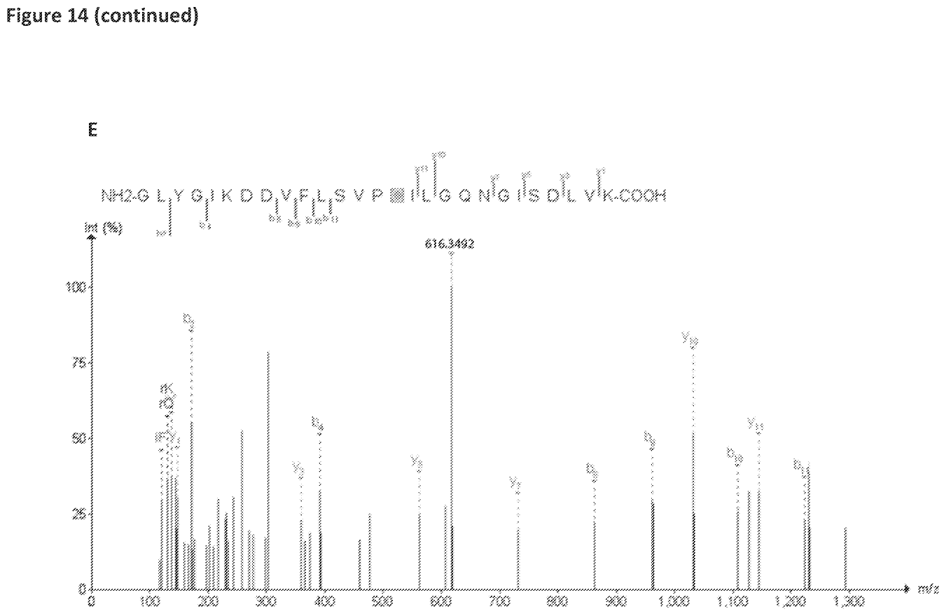

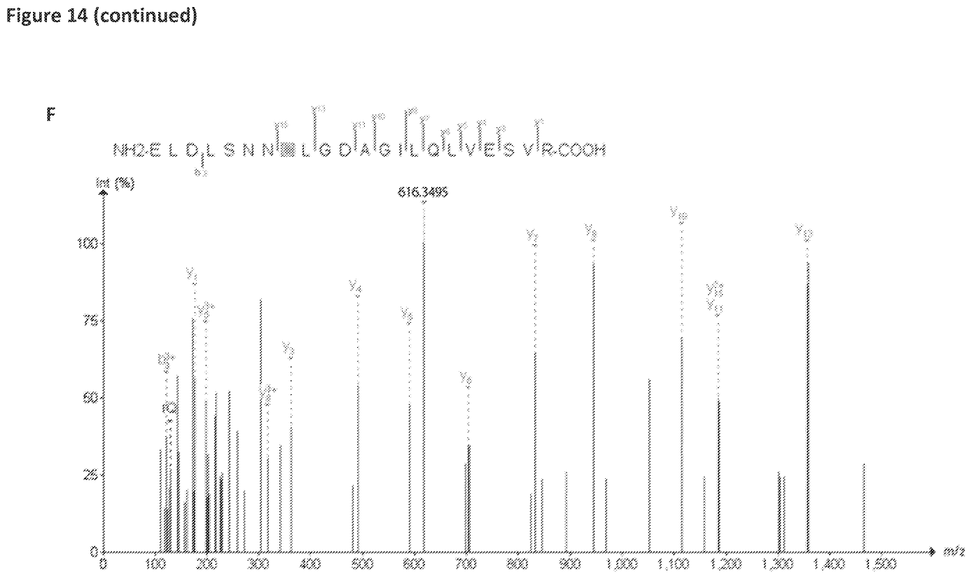

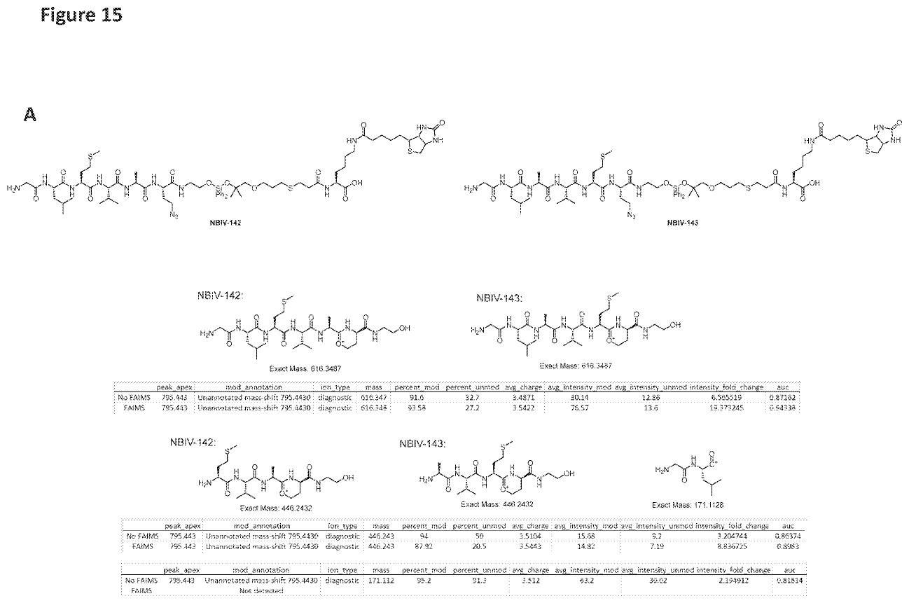

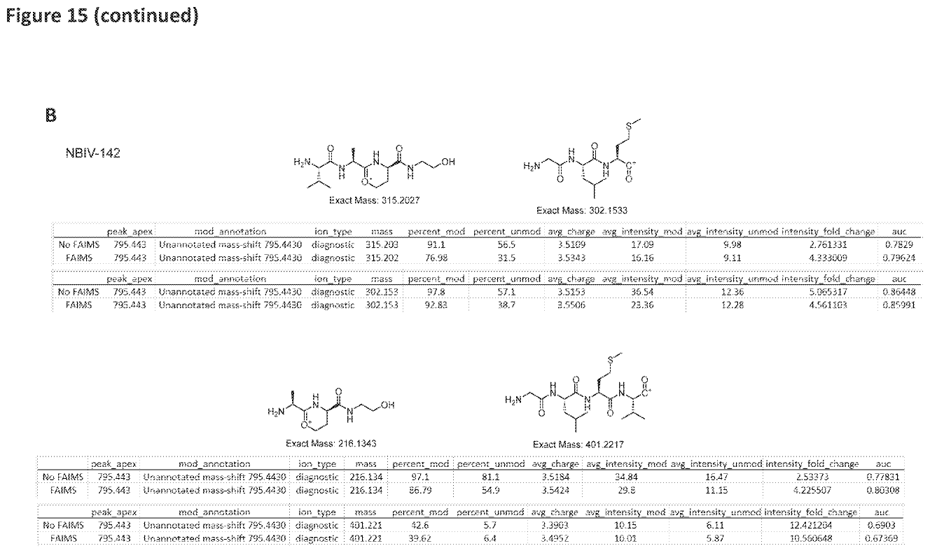

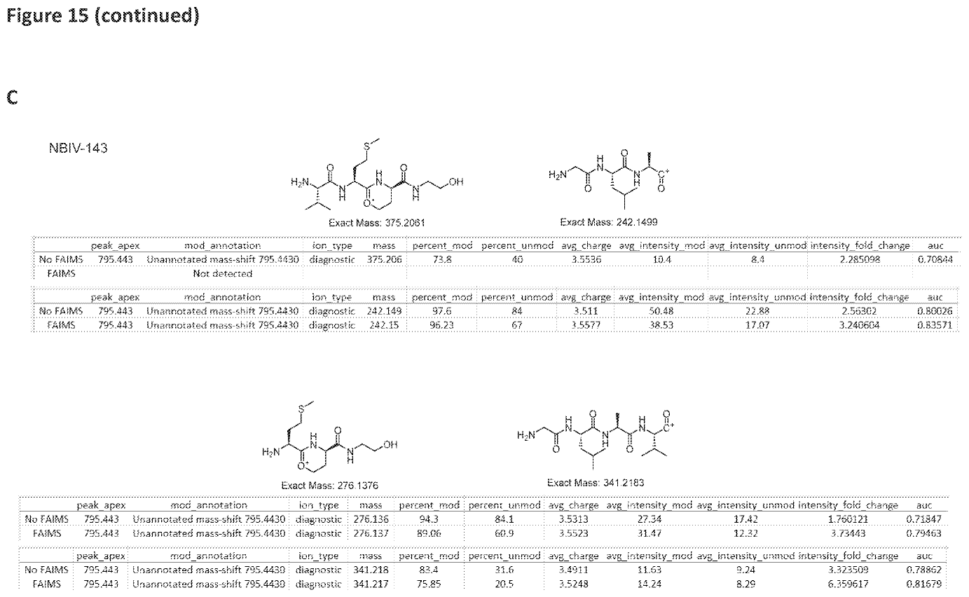

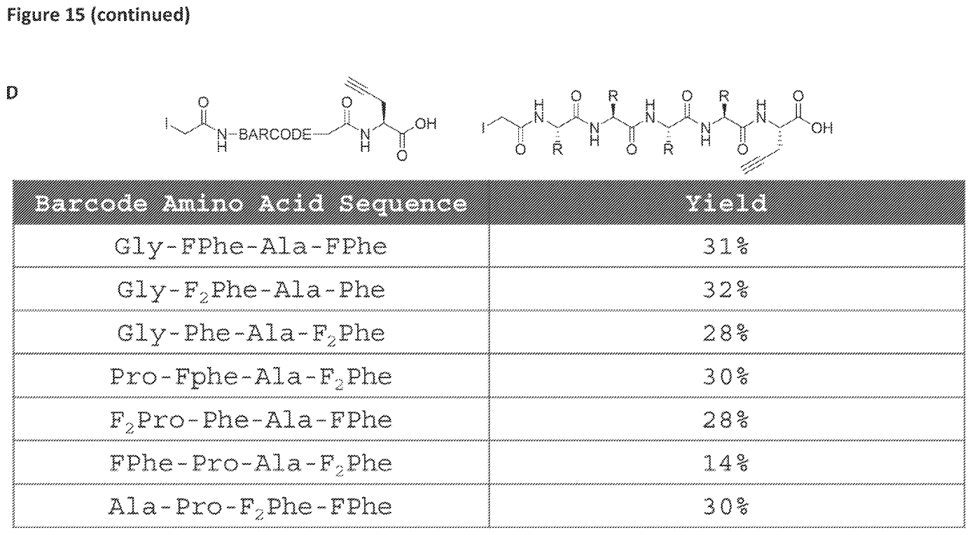

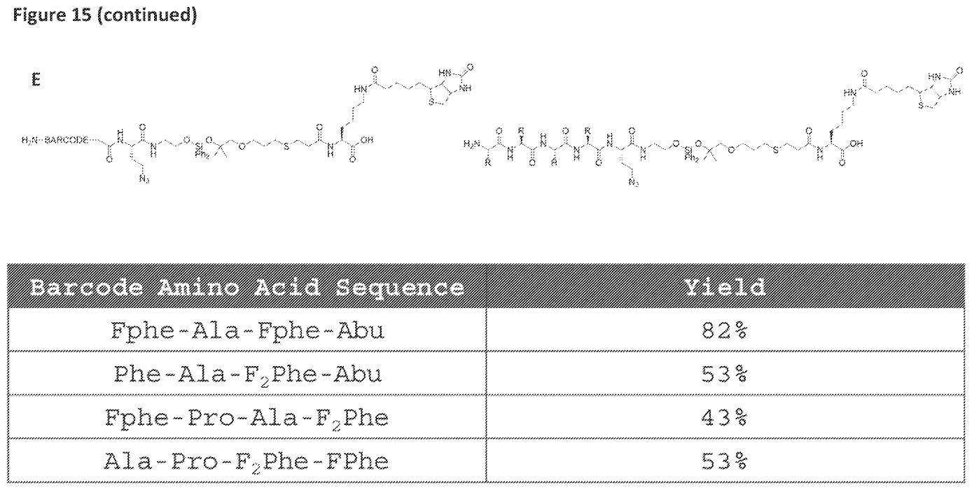

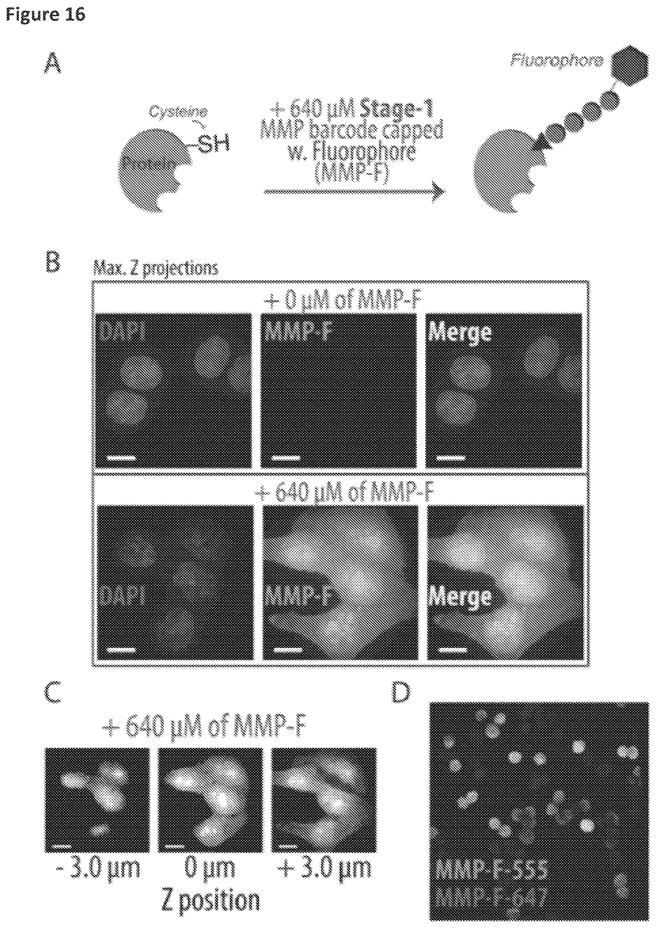

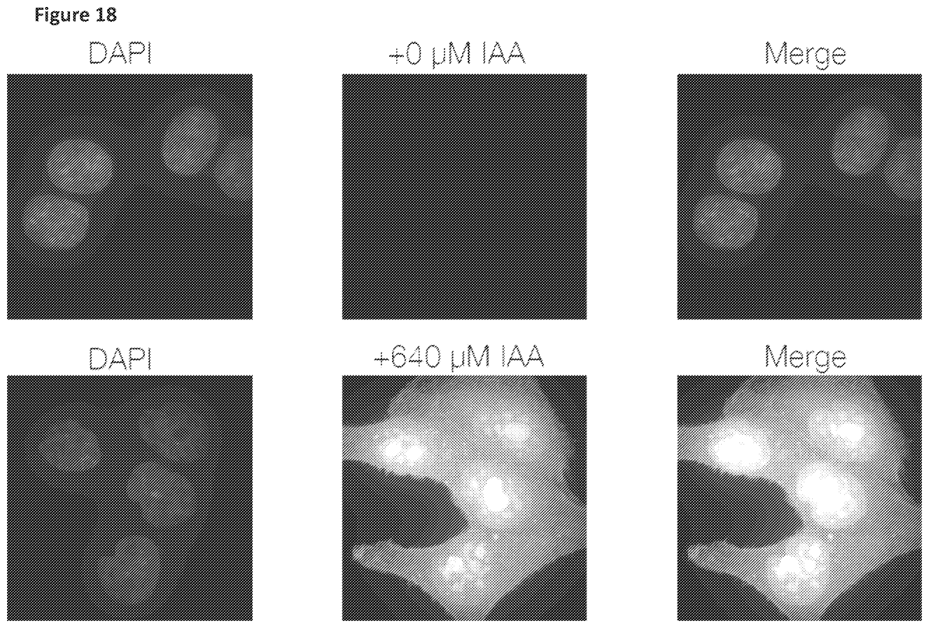

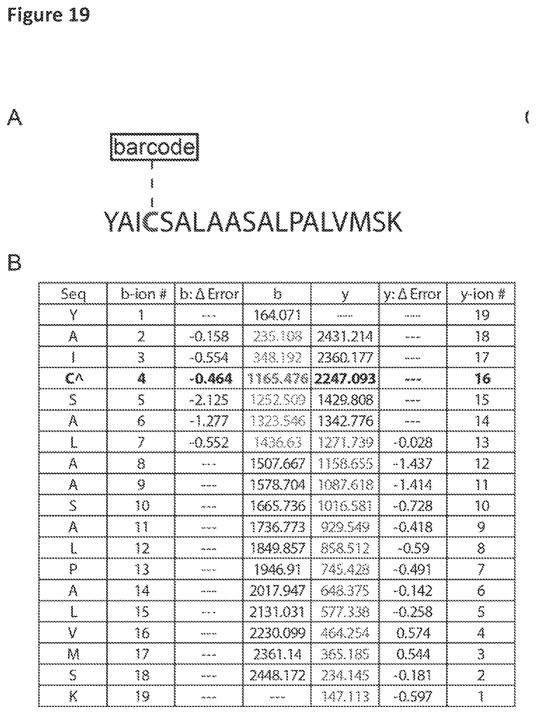

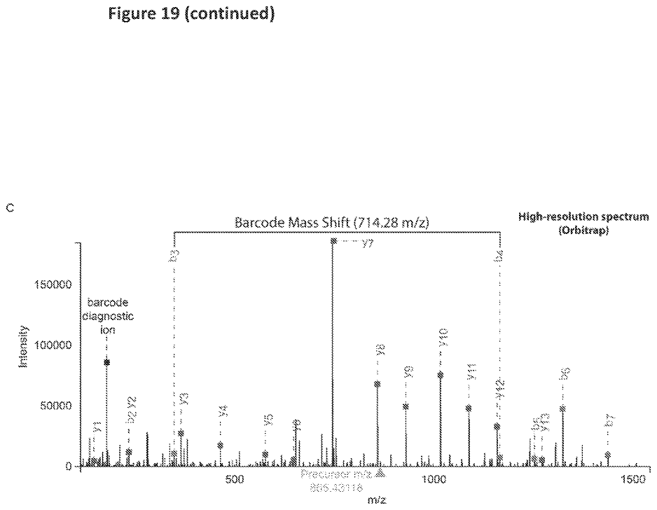

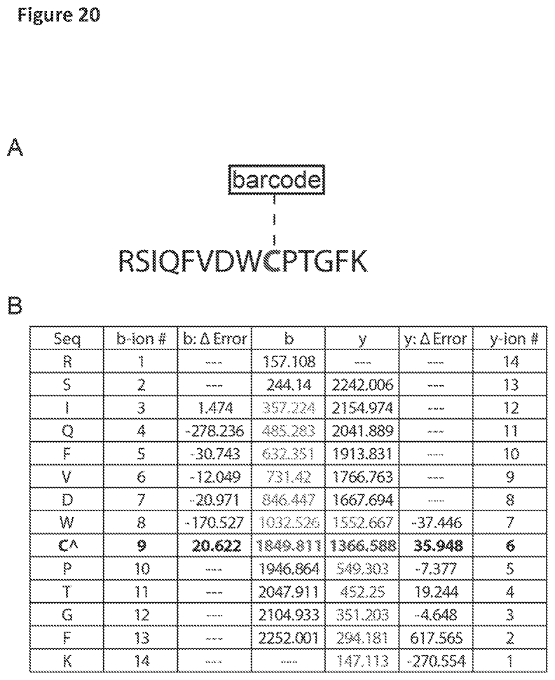

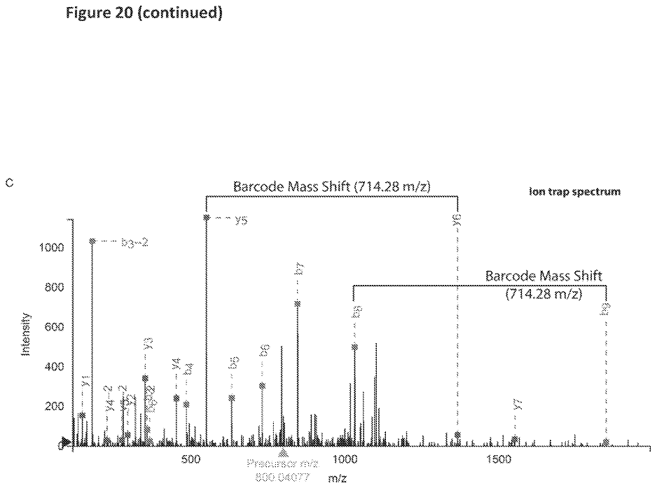

It was confirmed that peptide barcodes could be efficiently attached to proteins in situ (FIGS. 16-20). 5 amino-acid barcodes were engineered, with a click-reactive N-terminal azide, a 4 amino acid information carrying unit, and a terminal cysteine. Bulk cellular proteomes were first alkylated with iodoacetamide alkyne, enabling conjugation to the primary barcode. Near complete (~100%) reactivity with solvent-accessible cysteine residues (~80% of total cysteine residues) was observed in fixed human cells (FIG. 20, Table 1). Mass spectrometry of indexed peptides from digested proteins identified diagnostic fragment ions that enabled both peptide sequencing and deconvolution of individual barcodes (FIG. 20). This labeling was extended to a second round of barcode, again capping with iodoacetamide alkyne followed by click conjugation to a second barcode sequence. Lastly, the barcoded proteins were capped with desthiobiotin iodoacetamide or fluorophores (FIG. 8).

TABLE 1
Summary of mass spectrometry data from experiments in which an initial round of
barocde attachment via alkylation of cysteine residues (round 1) was followed a
second round of click chemistry (round 2) to further extend the affixed barcodes.

| | | | | # Alkylated + |
|---|---|---|---|---|
| | | # Cys-containg | # Alkylated | Clicked |
| | | proteins | proteins | proteins |
| Alkylating reagent | Click reagent | detected | detected | detected |
| Iodoacetamide-alkyne | Desthiobiotin-azide | 1803 | 1524 | 1465 |
| Iodoacetamide-alkyne | Desthiobiotin-azide | 1388 | 1112 | 1077 |
| Iodoacetamide-alkyne | Biotin-picolyl-azide | 1156 | 857 | 827 |
| Iodoacetamide-alkyne | SLGTC (peptide barcode) | 1546 | 1249 | 136 |
| | (SEQ ID NO: 21) | | | |

Implementation of in situ labeling and peptide recovery protocols that are compatible with combinatorial indexing by split-pooling. Starting from the preliminary tags, optimized protocols were developed to affix MMP barcodes to proteins in situ. Sample handling to minimize the loss of proteins was optimized. MMP's combinatorial indexing allows for pooling of individual cells to significantly reduce sample losses and boost sensitivity for peptide detection^{8,30}.

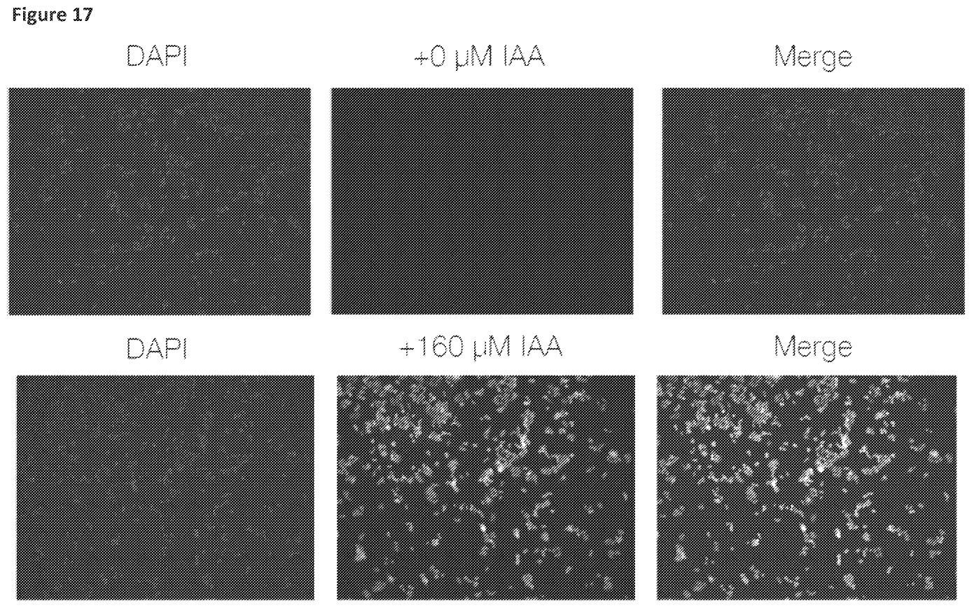

An imaging-compatible barcoding strategy using MMP barcodes was established (FIGS. 17-19) to monitor barcoding in fixed cells. Fluorescence-based capping of MMP barcodes allowed the quantitative monitoring of barcode attachment efficiency, sample penetration, and cellular mixing. This approach also allowed for monitoring and elimination of cell clumping, which is critical for the success of the combinatorial indexing strategy. HCT116 colorectal cancer cells were labeled with iodoacetamide alkyne followed by click barcoding using the schema in FIG. 4 and capping with a cysteine-reactive fluorophore. Importantly, minimal cell clumping was observed when using the optimized labeling and pooling protocols (FIGS. 16-18). Robust barcoding was apparent throughout the cells, including in the difficult to access nuclear and nucleolar compartments but was absent from controls (FIGS. 16-18).

### Example 2

Unify and automate cellular barcoding for MMP. To establish the combinatorial indexing by split pooling scheme, cells will be subjected to two rounds of barcoding using fluorescent barcode caps, allowing the careful monitoring of cell clumping, which can result in barcode collision due to clumped cells co-migrating to the same wells during multiple rounds of barcode addition and extension. In parallel, modern proteomics sample preparation techniques for cells and tissues that are compatible with 96-well format processing will be leveraged, including cell lysis, protein digestion to peptides, and sample cleanup^{31,32}, simplifying the implementation of the split pooling scheme for cellular indexing with the downstream workflows needed for proteomic sample processing.

Plate-based processing of up to 96 samples can be fully automated with functionality for fixation, solution-phase hybridization, cell lysis, protein digestion, reversing fixation, peptide cleanup, and other sample-handling needs. Automation of MMP methods will facilitate rapid optimization of ongoing MMP projects and provide the throughput necessary to process large cohorts of single-cell samples. High sample recovery and reproducibility for quantitative assays, can be achieved and monitored using fluorescence capping as shown in FIG. 17 tracking peptide and protein identifications in standard samples.

Engineer integrated isobaric barcodes that can support hundreds or more unique orderings for cellular indexing. Combinatorial indexing provides flexibility in the number of distinct barcode species that can be deployed per round and the length of the barcode used. These barcodes can be isobaric (same mass) to simplify the mass spectrometry methods needed to identify barcode sequences. Barcodes will be synthesized to allow for simultaneous decoding of each of their unique amino acid sequences. Fixed length barcodes can then be detected by the fragment ions released during mass spectrometric analyses The number of unique combinations C supported for barcodes of length L units that are added over N rounds is C=(L!)^{N} (FIG. 5B) 2). Thus, 4 amino acid barcodes (L=4) added in one round supports up to 4!=24 unique barcode combinations and (4!)²=576 in two rounds. Expanding to L=6 amino acids support up to 6!=720 in one round and up to (6!)²=518,400 in two. The diversity of amino acids provides an ample number of unique combinations that will enable highly parallelized screens of the cellular responses to perturbation. The number of single cells that can be profiled using MMP grows dramatically with even one round of barcode extension. Two rounds of L=4 amino acids generate 576 unique barcodes and two rounds of L=8 amino acids generate 1,625,702,400 unique barcodes. Notably, at the highest levels of sample multiplexing the number of unique barcodes would exceed the maximum number of ions that can be stabilized in current mass spectrometers (n~3,000,000 ions at charge=1+). In the mature form, the combinatorial complexity of tandem barcode addition will enable single-cell proteomics at the scale of entire tumor xenografts or patient biopsy samples.

Continue development of mass spectrometric and computational methods to detect and decode chemical barcodes in raw spectral data. The core concept driving MMP's dramatic increase in throughput relies on the development of robust mass spectrometry methods to simultaneously identify peptides and quantify the relative abundance of individual barcodes associated with each peptide. The instant mass spectrometric method will perform a first-of-its-kind two-stage data independent acquisition. In the first stage, peptides will be fragmented to enable sequencing. In the second stage, isobaric barcodes will be released and fragmented for decoding of the MMP index. Matched peptide and barcode elution profiles will be used to quantify the relative abundance of each peptide associated with each barcode and derive cell-specific quantitative proteomes. MMP's cell pooling (FIG. 4) will boost the ability to detect peptides from individual cells. While the barcode fragment ions will be unique, peptide fragment ions are additive because they are the same for cell to cell, i.e., an MMP pool of 100 cells would generate a 100-fold ‘boost’ in peptide fragment intensity compared to a single, non-multiplexed cell. Additionally, in the fragment spectra, the peptide fragment ions will be ~100-fold more intense than the individual barcode fragment ions making barcode identification and quantification very challenging. To address this, a targeted barcode detection analysis (targeted MS3) to quantify individual barcodes will be performed. Isobaric barcodes will be released by low energy fragmentation of triazole using beam-type collisional dissociation. Window specific isolation of these released barcodes will then enable robust quantification. Post hoc analysis tools for decoding and quantifying individual peptide and barcode intensities will be built using established, open-source proteomics codebases such as Comet and Skyline^{40,41}.

### REFERENCES

1. Zhang, Y. et al. Single-cell RNA sequencing in cancer research. J. Exp. Clin. Cancer Res. 40, 1-17 (2021).
2. Maltz, E. & Wollman, R. Quantifying the phenotypic information in mRNA abundance. Mol. Syst. Biol. 18, e11001 (2022).
3. Gatto, L. et al. Initial recommendations for performing, benchmarking and reporting single-cell proteomics experiments. Nat. Methods 20, 375-386 (2023).
4. MacCoss, M. J. et al. Sampling the proteome by emerging single-molecule and mass spectrometry methods. Nat. Methods 20, 339-346 (2023).
5. Johnston, S. M. et al. Rapid, One-Step Sample Processing for Label-Free Single-Cell Proteomics. J. Am. Soc. Mass Spectrom. (2023) doi: 10.1021/jasms.3c00159.
6. Mund, A. et al. Deep Visual Proteomics defines single-cell identity and heterogeneity. Nat. Biotechnol. 40, 1231-1240 (2022).
7. Petelski, A. A. et al. Multiplexed single-cell proteomics using SCOPE2. Nat. Protoc. 16, 5398-5425 (2021).
8. Budnik, B., Levy, E., Harmange, G. & Slavov, N. SCOPE-MS: mass spectrometry of single mammalian cells quantifies proteome heterogeneity during cell differentiation. Genome Biol. 19, 161 (2018).
9. Demaree, B. et al. Joint profiling of DNA and proteins in single cells to dissect genotype-phenotype associations in leukemia. Nat. Commun. 12, 1583 (2021).
10. Martin, B. K. et al. Optimized single-nucleus transcriptional profiling by combinatorial indexing. Nat. Protoc. 1-20 (2022).
11. Amini, S. et al. Haplotype-resolved whole-genome sequencing by contiguity-preserving transposition and combinatorial indexing. Nat. Genet. 46, 1343-1349 (2014).
12. Cao, J. et al. The single-cell transcriptional landscape of mammalian organogenesis. Nature 566, 496-502 (2019).
13. Rössler, S. L., Grob, N. M., Buchwald, S. L. & Pentolute, B. L. Abiotic peptides as carriers of information for the encoding of small-molecule library synthesis. Science 379, 939-945 (2023).
14. Backus, K. M. et al. Proteome-wide covalent ligand discovery in native biological systems. Nature 534, 570-574 (2016).
15. Kuljanin, M. et al. Reimagining high-throughput profiling of reactive cysteines for cell-based screening of large electrophile libraries. Nat. Biotechnol. 39, 630-641 (2021).
16. Xiao, H. et al. A Quantitative Tissue-Specific Landscape of Protein Redox Regulation during Aging. Cell 180, 968-983.e24 (2020).
17. Darabedian, N. et al. Depletion of creatine phosphagen energetics with a covalent creatine kinase inhibitor. Nat. Chem. Biol. 19, 815-824 (2023).
18. Gygi, S. P. et al. Quantitative analysis of complex protein mixtures using isotope-coded affinity tags. Nat. Biotechnol. 17, 994-999 (1999).
19. Ma, T. P. et al. AzidoTMT Enables Direct Enrichment and Highly Multiplexed Quantitation of Proteome-Wide Functional Residues. J. Proteome Res. 22, 2218-2231 (2023).
20. Burton, N. et al. A solid-phase compatible silane-based cleavable linker enables custom isobaric quantitative chemoproteomics. ChemRxiv (2023) doi: 10.26434/chemrxiv-2023-n8h29.
21. Yan, T. et al. Enhancing Cysteine Chemoproteomic Coverage through Systematic Assessment of Click Chemistry Product Fragmentation. Anal. Chem. 94, 3800-3810 (2022).
22. Cusanovich, D. A. et al. Multiplex single cell profiling of chromatin accessibility by combinatorial cellular indexing. Science 348, 910-914 (2015).
23. Cao, J. et al. Comprehensive single-cell transcriptional profiling of a multicellular organism. Science 357, 661-667 (2017).
24. Ramani, V. et al. Massively multiplex single-cell Hi-C. Nat. Methods 14, 263-266 (2017).
25. Jovic, D. et al. Single-cell RNA sequencing technologies and applications: A brief overview. Clin. Transl. Med. 12, e694 (2022).
26. Polasky, D. A. et al. MSFragger-Labile: A Flexible Method to Improve Labile PTM Analysis in Proteomics. Mol. Cell. Proteomics 22, 100538 (2023).
27. Geiszler, D. J., Polasky, D. A., Yu, F. & Nesvizhskii, A. I. Detecting diagnostic features in MS/MS spectra of post-translationally modified peptides. Nat. Commun. 14, 4132 (2023).
28. Li, J. et al. TMTpro reagents: a set of isobaric labeling mass tags enables simultaneous proteome-wide measurements across 16 samples. Nat. Methods 17, 399-404 (2020).
29. Schweppe, D. K. et al. Mitochondrial protein interactome elucidated by chemical cross-linking mass spectrometry. Proc. Natl. Acad. Sci. U.S.A 114, 1732-1737 (2017).
30. Bennett, H. M., Stephenson, W., Rose, C. M. & Darmanis, S. Single-cell proteomics enabled by next-generation sequencing or mass spectrometry. Nat. Methods 20, 363-374 (2023).
31. Desai, H. S. et al. SP3-Enabled Rapid and High Coverage Chemoproteomic Identification of Cell-State-Dependent Redox-Sensitive Cysteines. Mol. Cell. Proteomics 21, (2022).
32. Gaun, A. et al. Automated 16-Plex Plasma Proteomics with Real-Time Search and Ion Mobility Mass Spectrometry Enables Large-Scale Profiling in Naked Mole-Rats and Mice. J. Proteome Res. 20, 1280-1295 (2021).
33. Boatner, L. M., Palafox, M. F., Schweppe, D. K. & Backus, K. M. CysDB: a human cysteine database based on experimental quantitative chemoproteomics. Cell Chem Biol 30, 683-698.e3 (2023).
34. Yan, T. et al. SP3-FAIMS chemoproteomics for high-coverage profiling of the human cysteinome. Chembiochem 22, 1841-1851 (2021).
35. Keele, G. R. et al. Global and tissue-specific aging effects on murine proteomes. Cell Rep. 42, 112715 (2023).
36. Schweppe, D. K. et al. Full-Featured, Real-Time Database Searching Platform Enables Fast and Accurate Multiplexed Quantitative Proteomics. J. Proteome Res. 19, 2026-2034 (2020).
37. McGann, C. D. et al. Real-time spectral library matching for sample multiplexed quantitative proteomics. bioRxiv (2023) doi: 10.1101/2023.02.08.527705.
38. Yu, Q. et al. Benchmarking the Orbitrap Tribrid Eclipse for Next Generation Multiplexed Proteomics. Anal. Chem. 92, 6478-6485 (2020).
39. Yu, F. et al. Analysis of DIA proteomics data using MSFragger-DIA and FragPipe computational platform. Nat. Commun. 14, 4154 (2023).
40. Chavez. J. D. et al. A General Method for Targeted Quantitative Cross-Linking Mass Spectrometry. PLoS One 11, e0167547 (2016).
41. O'Brien, J. J. et al. Conditional fragment ion probabilities improve database searching for nonmonoisotopic precursors. J. Proteome Res. 22, 334-342 (2023).

1. A functionalized label, comprising a peptide of 3-20 amino acids in length, wherein the peptide is covalently bound to
(a) one or more azide molecules,
(b) one or more alkyne molecules, or
(c) one or more azide molecules and one or more alkyne molecules.

2. The functionalized label of claim 1, wherein
the one or more azide molecules comprises the molecular formula N₃, and/or
the one or more alkyne molecules comprises iodoacteamide alkyne, cycloalkyne, cyclooctyne, iodoacetamide alkyne, or alkyne NHS ester.

3. The functionalized label of claim 1, wherein the one or more alkyne molecules comprises a cysteine-capping alkyne molecule, wherein the cysteine-capping alkyne molecule comprises biotin, desthiobiotin iodoacetamide (DBIA), iodoacetimide, cysteine-reactive phosphate tags (CPT), N-ethylmaleimide (NEM), chloroacetamide, sulfonyl substituted heterocycles, iodoacetamide alkyne, or an azide.

4. The functionalized label of claim 1, wherein the peptide comprises a cysteine, and/or further comprises one or more functional moieties.

5. (canceled)

6. The functionalized label of claim 4, wherein the peptide further comprises one or more functional moieties, wherein the one or more functional moieties comprises streptavidin, a hexahistidine tag, biotin, Desthiobiotin, a HA tag, a FLAG tag, phosphonate, a florescent probes, a fluorophore, an isotopic labelling probe, or a metal-based labelling probe.

7. The functionalized label of claim 1, further comprising a cleavable linker, wherein the cleavable linker comprises dialkoxydiphenylsilane (DADPS).

8. The functionalized label of claim 1, wherein the peptide is 3-19, 3-18, 3-17, 3-16, 3-15, 3-14, 3-13, 3-12, 3-11, or 3-10 amino acids in length, or wherein the peptide is 4-20, 4-19, 4-18, 4-17, 4-16, 4-15, 4-14, 4-13, 4-12, 4-11, 4-10, 5-20, 5-19, 5-18, 5-17, 5-16, 5-15, 5-14, 5-13, 5-12, 5-11, 5-10, 6-20, 6-19, 6-18, 6-17, 6-16, 6-15, 6-14, 6-13, 6-12, 6-11, or 6-10 amino acids in length.

9. The functionalized label claim 1, comprising:
(a) a peptide of 3-20 amino acids in length;
(b) a branched azide molecule covalently linked to a carboxy terminus of the peptide;
(c) a cleavable linker covalently linked to the branched azide molecule;
(d) a biotin moiety covalently linked to the cleavable linker; and
(e) an alkyne molecule covalently linked to the biotin moiety.

10. A labeled protein comprising a first functionalized label according to claim 1, bound to the protein, wherein the first functionalized label comprises a first peptide.

11. A labeled protein comprising:
(a) a first functionalized label according to claim 1, bound to the protein, wherein the first functionalized label comprises a first peptide; and
(b) a second functionalized label according to claim 1, wherein the second functionalized label comprises a second peptide, and wherein the second functionalized label is covalently bound to the first functionalized label.

12. A labeled protein comprising:
(a) a first functionalized label according to claim 1, bound to the protein, wherein the first functionalized label comp es a first peptide;
(b) a second functionalized label according to claim 1, wherein the second functionalized label comprises a second peptide, and wherein the second functionalized label is covalently bound to the first functionalized label; and
(c) one or more additional functionalized labels according to claim 1, wherein each additional functionalized label comprises an additional peptide, and wherein each additional functionalized label is covalently bound to a functionalized label present in the composition or the protein.

13. The labeled protein of claim 10, wherein the first functionalized label is
(i) covalently bound directly to the protein via binding of one or more alkyne molecule to a cysteine residue in the protein, or
(ii) covalently bound to an iodoacetamide, wherein the iodoacetamide is covalently bound to a cysteine residue in the protein.

14. The labeled protein of claim 11, wherein covalent binding of the functionalized labels to each other occurs via binding of one or more azide molecule on one functionalized label to one or more alkyne molecule on another functionalized label.

15. The labeled protein of claim 12, wherein the first peptide, the second peptide, and the one or more additional peptide all have a different amino acid sequence; the same amino acid sequence; and/or are isobaric.

16-17. (canceled)

18. A method of labeling a protein comprising:
(a) contacting a protein with a first functionalized label to produce a single labeled protein, wherein the first functionalized label comprises a functionalized label of claim 1, wherein the peptide comprises a first peptide; and
(b) contacting the single labeled protein with a second functionalized label to produce a double labeled protein, wherein the first functionalized label covalently binds to the second functionalized label, and
wherein the second functionalized label comprises a functionalized label of claim 1 wherein the peptide comprises a second peptide.

19-30. (canceled)

31. A method comprising:
(a) dividing a biological sample, comprising cells comprising individual proteins, into a plurality of separate first samples;
(b) contacting each separate first sample with a different first functionalized label according to claim 1, wherein each different first functionalized label comprises a different first peptide, wherein the contacting is carried out under conditions wherein each first functionalized label covalently binds to an individual protein in a plurality of proteins in the first sample that it is contacted with;
(c) combining the plurality of separate first samples to generate a combined sample;
(d) dividing the combined sample into a plurality of separate second samples;
(e) contacting each separate second sample with a different second functionalized label according to claim 1, wherein each different second functionalized label comprises a different second peptide, wherein the contacting is carried out under conditions wherein each second functionalized label covalently binds to the first functionalized label on the individual protein in the plurality of proteins in the second sample that it is contacted with;
to produce a labeled plurality of proteins in a labeled biological sample;
wherein the labeled plurality of proteins comprises a plurality of individual proteins from each individual cell in the biological sample and wherein each plurality of individual proteins from each individual cell is bound to a unique sequence of first and second functionalized labels.

32-49. (canceled)

50. A composition, comprising a peptide of 3-20 amino acids in length, wherein the peptide is covalently bound to (a) one or more azide molecules or (b) one or more alkyne molecules.

51-55. (canceled)

56. A method comprising:
a. dividing a biological sample into a plurality of separate first samples;
b contacting each separate first sample with a different first label, wherein each of the first labels binds to a plurality of proteins in each of the plurality of separate first samples;
c. combining the plurality of separate first samples to generate a combined sample;
d. dividing the combined sample into a plurality of separate second samples;
e. contacting each separate second sample with a different second label, wherein each of the second labels binds to the first label on the plurality of proteins in each of the plurality of separate second samples; and
wherein the plurality of proteins from each individual cell in a biological sample is bound to a unique sequence of first and second labels.

57-75. (canceled)
